(19) United States
(12) Patent Application Publication
(10) Pub. No.: US 2026/0073516 A1
(43) Pub. Date: Mar. 12, 2026

(54) DEVICE AND METHOD FOR NON-INVASIVE AND NON-CONTACT PHYSIOLOGICAL WELL BEING MONITORING AND VITAL SIGN ESTIMATION

(71) Applicant: BRGX Technologies LLC, Chattanooga, TN (US)

(72) Inventors: Julian Gerald Dcruz, Kollam (IN); Ted Huang, Taipei (TW); Pai-chang Yeh, Zhubei (TW)

(73) Assignee: BRGX Technologies LLC, Chattanooga, TN (US)

(21) Appl. No.: 19/342,617

(22) Filed: Sep. 28, 2025

Related U.S. Application Data
(63) Continuation-in-part of application No. 17/729,523, filed on Apr. 26, 2022.
(63) Continuation-in-part of application No. 17/645,984, filed on Dec. 25, 2021.
(63) Continuation-in-part of application No. 19/081,519, filed on Mar. 17, 2025.

(51) Int. Cl. G06T 7/00; A61B 5/00; A61B 5/024; A61B 5/11; A61B 5/1455; A61B 5/318; G06V 10/141; G06V 10/82; G06V 40/16; G16H 40/67; G16H 50/20

(52) U.S. Cl. CPC G06T 7/0012; A61B 5/02427; A61B 5/1102; A61B 5/1128; A61B 5/14551; A61B 5/318; A61B 5/742; G06V 10/141; G06V 10/82; G06V 40/166; G06V 40/171; G16H 40/67; G16H 50/20; G06T 2207/20084; G06T 2207/30004; G06T 2207/30201

(57) ABSTRACT

A non-contact, non-invasive health monitoring device and method utilizing advanced artificial intelligence (AI) and machine learning techniques. This system captures real-time image data of a user's face using a high-resolution camera and processes the data to extract physiological signals, including Photoplethysmography (PPG, iPPG, and rPPG) and Ballistocardiography (BCG and iBCG). By leveraging facial landmark detection and deep learning models such as Convolutional Neural Networks (CNNs) and Transformers, the device predicts vital signs such as heart rate, respiratory rate, blood pressure, and oxygen saturation, alongside wellness metrics like stress levels and metabolic health. The device employs robust feature construction and signal processing modules to ensure accurate metrics under varying conditions, with error margins below 5%. Outputs are displayed in real time and integrated with external systems using standardized healthcare protocols. Applications include telehealth, fitness monitoring, public health screening, and automotive safety systems.

### RELATED APPLICATION

This application claims the benefit of U.S. patent application Ser. No. 19/081,519, titled DEVICE AND METHOD FOR NON-INVASIVE AND NON-CONTACT PHYSIOLOGICAL WELL BEING MONITORING AND VITAL SIGN ESTIMATION, filed Mar. 17, 2025, U.S. patent application Ser. No. 17/729,523, titled “SYSTEM, METHOD AND APPARATUS FOR NON-INVASIVE & NON-CONTACT MONITORING OF HEALTH CHARACTERISTICS USING ARTIFICIAL INTELLIGENCE (AI)”, filed on Apr. 26, 2022, and to U.S. patent application Ser. No. 17/645,984, titled “APPARATUS, METHOD AND DEVICE FOR NON-CONTACT AND NON-INVASIVE BLOOD SUGAR MONITORING TO HELP MONITOR DIABETIC PATIENTS AND HYPERCOAGULATION”, filed on Dec. 25, 2021. These patent applications are incorporated herein by reference.

### TECHNICAL FIELD

The present invention is of a system and method for to non-contact and non-invasive health monitoring determined by analyzing physiological signals from imaging data and using deep learning techniques to estimate vital signs and wellness parameters. This present invention is particularly applicable to health assessment, fitness monitoring, telehealth, public health screening, and advanced driver assistance systems (ADAS). We give three examples of the advantages of this invention in real life—monitoring of truck drivers in real time by a trucking company, detecting spoofing, and in monitoring individuals for signs of nervousness and other often terrorist- or smuggler-related facial readings—but these three examples are not meant to be limiting to the full extent of uses of this invention.

### BACKGROUND

The following description includes information that may be useful in understanding the present invention. It is not an admission that any of the information provided herein is prior art or relevant to the presently claimed subject matter, or that any publication specifically or implicitly referenced is prior art.

Recent advancements in artificial intelligence (AI) and machine learning have significantly enhanced the capabilities of health monitoring devices, particularly in non-invasive and non-contact methods. Traditional health monitoring devices often require physical contact through wearable sensors or direct physiological measurements, which can cause discomfort and inconvenience for users. These conventional methods may also face limitations in accessibility, particularly in contexts where physical contact is impractical, such as during pandemics, in remote locations, or areas with limited healthcare infrastructure. As a result, there is a growing demand for non-contact solutions that can offer accurate, real-time monitoring without physical interaction.

Conventional non-invasive vital-sign monitors, such as pulse-oximeter devices, estimate arterial oxygen saturation (SpO₂) and pulse rate by transmitting and receiving light of at least two wavelengths (typically red and infrared) through perfused tissue, most commonly a fingertip or earlobe. A ratio-of-ratios computation of the detected optical intensities yields an SpO₂ value, while the temporal periodicity of the photoplethysmographic waveform (PPG) provides the pulse rate. Although pulse oximeters are inexpensive and clinically accepted, they require direct sensor-to-skin contact, are sensitive to motion artefacts and ambient-light interference, and do not report additional hemodynamic metrics such as blood-pressure trends or heart-rate variability (HRV).

Advances in computer vision and digital signal processing have enabled “remote” photoplethysmography (rPPG), in which a conventional red-green-blue (RGB) camera acquires small intensity changes in facial skin that correspond to pulsatile blood-volume variations. Early rPPG algorithms applied spatial averaging and band-pass filtering to extract a pulsatile waveform from the green channel, followed by peak detection to estimate heart rate. Recent developments in machine learning have extended this concept to imaging photoplethysmography (iPPG), wherein deep neural networks learn spatio-temporal representations directly from video frames, thereby improving signal-to-noise ratio, suppressing motion artefacts, and permitting estimation of additional parameters, including respiratory rate and perfusion index. Consequently, iPPG furnishes a fully contact-free modality that derives continuous physiological waveforms from successive image frames, delivering a camera-based alternative that is inherently more comfortable for the user, readily deployable on commodity devices, and resilient to the ambient-light variations typical of everyday environments.

While PPG-derived techniques accurately capture pulse-related blood-volume changes, they do not fully characterise the mechanical activity of the heart. Clinical cardiac assessment traditionally relies on electrocardiography (ECG), which measures bioelectric potentials via adhesive electrodes placed on the torso or limbs. ECG electrodes, however, still require skin preparation and physical attachment, restricting long-term or opportunistic use.

Ballistocardiography (BCG) quantifies minute body accelerations caused by the recoil of blood ejection during each cardiac cycle. Historically, BCG was recorded with mechanical bed platforms or high-sensitivity force sensors embedded in weighing scales; more recently it has been measured by micro-electromechanical-system (MEMS) accelerometers placed under mattresses or inside wearable devices. BCG waveforms contain J-, H-, and I-peaks that correlate with stroke volume, cardiac output, and systolic time intervals, offering a pathway to estimate blood pressure and HRV without direct arterial measurement.

Emerging computer-vision methods now enable video-based BCG (iBCG). By magnifying sub-pixel facial displacements and tracking optical-flow vectors at high temporal resolution, machine-learning models can isolate repetitive micro-motions synchronous with cardiac ejection. The resulting iBCG waveform complements the simultaneously acquired iPPG signal: the former encodes mechanical events (aortic valve opening, blood acceleration), whereas the latter encodes volumetric hemodynamics.

Individually, imaging photoplethysmography (iPPG) supplies a continuous, high-temporal-resolution waveform that represents peripheral blood-volume changes; peak-to-peak intervals yield pulse rate, spectral content yields respiratory rate, and pulse-wave amplitude variations correlate with vasomotor tone and perfusion index. Because light absorption in cutaneous tissue varies with blood oxygenation, multispectral iPPG further permits inference of arterial oxygen saturation without physical contact.

Imaging ballistocardiography (iBCG), in contrast, captures minute inertial displacements of cranio-facial tissue that coincide with ventricular ejection, aortic recoil, and diastolic filling. Characteristic J-, H-, and I-waves permit estimation of stroke volume, pre-ejection period, and left-ventricular ejection time-mechanical indices that underlie blood-pressure dynamics and heart-rate variability (HRV).

Because iPPG encodes volumetric hemodynamics while iBCG encodes mechanical cardiac kinetics, the two modalities are complementary. Fusing their respective feature sets therefore reduces estimation ambiguity, improves robustness to motion artefacts, and enlarges the measurable vital-sign repertoire.

The fusion strategy draws inspiration from optical coherence tomography (OCT), in which multiple depth-resolved optical slices are co-registered and aggregated to reconstruct a high-fidelity vascular image. Analogously, the disclosed system employs a multi-branch neural network in which: (i) a first branch extracts spatial-temporal iPPG features from successive facial frames; (ii) a second branch extracts kinematic iBCG features from sub-pixel optical-flow fields; and (iii) a contextual branch processes static facial-frame features such as skin tone, illumination, and pose. Feature volumes from all branches are concatenated along a channel dimension and passed through depth-wise convolutional and self-attention layers that learn cross-modal correlations, yielding a fused cardiovascular representation suitable for real-time inference.

Empirical evaluation shows that the fused representation supports inference latencies below 50 ms on a commodity mobile-GPU and maintains signal-quality indices above 0.90 during head rotations of ±30 degrees and illuminance changes from 50 lx to 1 000 lx. Accordingly, the system yields rapid, motion-resilient estimates of systolic and diastolic blood pressure, pulse-transit time, beat-to-beat heart-rate variability, respiratory rate, and arterial oxygen saturation using only an RGB camera.

The derived vital signs form inputs to a wellness-analytics module that maps physiological state to higher-order wellness indicators. A correlational bio-computational scoring engine—trained on longitudinal datasets linking wearable-derived lifestyle logs, and clinician-verified outcomes—calculates: (i) acute stress index from sympathetic/parasympathetic HRV ratios; (ii) metabolic-health score from resting heart-rate trends, blood-pressure variability, and breathing patterns; (iii) immune-response readiness from combined HRV suppression and pulse-wave amplitude anomalies; and (iv) micronutrient-deficiency likelihood from long-term deviations in cardiopulmonary baselines. These wellness parameters provide users and clinicians with actionable insight that extends beyond raw physiology to an integrated assessment of overall health status.

Accordingly, the present disclosure combines camera-derived iPPG and iBCG sensing with OCT-inspired deep-feature fusion and wellness analytics to deliver a comprehensive, contact-free platform for continuous vital-sign monitoring and personalised health assessment. The disclosed low-latency, contact-free platform is expressly configured for deployment in dynamic operating environments that demand uninterrupted physiological surveillance. Illustrative use-case domains include: (i) driver-state modules integrated within advanced driver-assistance systems (ADAS) of autonomous and semi-autonomous vehicles, where continuous estimation of alertness, sympathetic stress load, and incipient cardiovascular anomalies enables adaptive safety interventions; (ii) operator-safety systems for heavy-equipment, transport-truck, crane, and construction-machinery personnel, in which real-time hemodynamic monitoring can forestall fatigue-related accidents; (iii) security checkpoints that combine liveness detection with physiological verification to counter presentation attacks and deep-fake imagery; and (iv) remote-care hubs for elderly, chronically ill, or post-operative patients, permitting clinicians to track hemodynamic stability without the burden of adhesive electrodes or wearable devices.

The same non-contact technology extends to broader public-health and consumer-wellness applications. Camera-based screening stations situated in high-traffic venues—airports, schools, workplaces, and sporting arenas—can perform rapid, contact-free triage of febrile or hemodynamically compromised individuals. Fitness enthusiasts and elite athletes may employ continuous video-based monitoring during exercise to quantify training load, recovery status, and autonomic balance, while telehealth platforms can deliver cardiopulmonary surveillance to patients in rural or underserved regions with no specialised hardware beyond a commodity camera. Accordingly, a need exists for a non-invasive, contact-free system that delivers rapid, motion-resilient, and clinically accurate estimation of vital signs and wellness metrics across automotive, industrial, security, healthcare, and public settings; the present disclosure satisfies this need through the synergistic fusion of camera-derived iPPG and iBCG signals processed via an OCT-inspired deep-learning architecture.

### SUMMARY

This summary is provided to introduce concepts related to a non-contact, non-invasive health monitoring device and method for monitoring a user's health characteristics using advanced machine learning techniques. The concepts are further described below in the detailed description. This summary is not intended to identify key features or essential features of the claimed subject matter, nor is it intended to be used to limit the scope of the claimed subject matter.

The present disclosure envisages a non-contact, non-invasive health monitoring device. The device includes a camera, a signal processing unit, a prediction unit, an output unit, and a communication interface. The camera is configured to capture real-time image data of a user's face, where the image data including a sequence of frames recorded under a set of conditions that ensure consistent pixel values. The signal processing unit is operatively coupled to the camera. The signal processing unit receives the sequence of frames from the camera. The signal processing unit comprises a video processor, a machine learning accelerator, and a feature construction module. The video processor is implemented as a dedicated hardware circuitry and is configured to detect facial landmarks using a predefined facial landmark detection algorithm and isolate one or more regions of interest (ROIs) based on the detected landmarks to build a time-series sequence of ROI data, where each ROI corresponds to a specific anatomical region. The machine learning accelerator is implemented as a specialized integrated circuit and is configured to receive the time-series sequence of ROI image data from the video processor, and apply a trained neural network model, custom trained on annotated video datasets correlated with ground-truth physiological signals including: electrocardiogram (ECG), and pulse oximetry data, to extract Photoplethysmography (iPPG) and Ballistocardiograph (iBCG) signals, representing subtle changes in pixel intensity linked to blood volume pulsations and micro-motions of facial tissue from the temporal and spatial intensity variations within the time-series sequence of ROI data. The feature construction module following the principles of optical coherence tomography (OCT) for feature preparation, providing improved background noise and motion reduction allowing better stability of feature extraction in dynamic environments of constant motion, wherein the feature construction module is implemented in hardware logic. The feature construction module is configured to combine the extracted iPPG and iBCG signals from the machine learning accelerator, with facial image features derived from the time-series sequence of ROI image data, wherein said facial image features comprise localized pixel intensity gradients calculated by comparing pixel values within and around the ROI landmarks, thus capturing spatial patterns of intensity variation, and construct a high-dimensional feature representation in the form of a volumetric tensor, where one dimension corresponds to the temporal progression across the frames, effectively treating time as a “depth” dimension, two dimensions represent the spatial coordinates of the ROI, capturing height and width, and one or more additional feature channels represent iPPG, iBCG, alongside the localized pixel intensity gradients. The prediction unit includes a hardware-based inference engine interfaced to the signal processing unit and is configured to receive the high-dimensional feature representation in the form of a volumetric tensor from the feature construction module; and apply a second trained neural network model, the second trained neural network model is trained on facial video data and corresponding ground-truth physiological measurements, and is made up of a combination of Convolutional layers, and Transformer with self-attention with positional encoding, specialized in analysing the volumetric tensor, combining temporal, spatial, and physiological feature relationships of the iPPG, iBCG and ROI data of volumetric tensor, to compute at least one physiological metric, with significant improvement in performance with error percentage less than 5% that aligns with medically validated criteria. The output unit comprises a hardware-based display interface configured to display the predicted at least one physiological metric from the prediction unit, in real-time to a user, providing immediate feedback on the subject's health status. The output unit further comprises a hardware-based probabilistic inference component configured to estimate an uncertainty metric associated with the predicted at least one physiological metric by applying a statistically grounded method, and inference using a trained ensemble of models that provides variance estimates, to yield a quantifiable confidence measure indicating the reliability of the prediction. The communication interface is implemented as a hardware module and is configured to transmit the predicted at least one physiological metric and its associated uncertainty metric to an external device or networked system via a standardized communication protocol; and format the transmitted data following standard healthcare data interchange protocols for ensuring compatibility with electronic health record systems or cloud-based analytics platforms.

The present disclosure further envisages a non-contact, non-invasive health monitoring device. The device includes a camera, a signal processing unit, a prediction unit, an output unit, and a communication interface. The camera configured to capture a real-time digital image data of a subject's face. The signal processing unit is operatively coupled to the camera. The signal processing unit receives the real-time digital image data. The signal processing unit comprises video processor, a machine learning accelerator, and a feature construction module. The video processor is implemented as dedicated hardware circuitry and is configured to detect facial landmarks using a predefined facial landmark detection algorithm, and isolate one or more regions of interest (ROIs) based on the detected landmarks to build a real-time series sequence of ROI data, wherein each ROI corresponds to a specific anatomical region. The machine learning accelerator is implemented as a specialized integrated circuit and is configured to receive the real time-series sequence of ROI data from the video processor; and apply a trained neural network mode to the real-time series sequences to extract Photoplethysmography (iPPG) and Ballistocardiograph (iBCG) signals. The feature construction module following the principles of optical coherence tomography (OCT) for feature preparation, providing improved background noise and motion reduction allowing better stability of feature extraction in dynamic environments of constant motion, wherein the feature construction module is implemented in hardware logic. The feature construction module is, configured to combine the extracted iPPG and iBCG signals from the machine learning accelerator, with facial image features derived from the real-time-series sequence of ROI data, wherein said facial image features comprise localized pixel intensity gradients calculated by comparing pixel values within and around the ROI landmarks, thus capturing spatial patterns of intensity variation; and construct a high-dimensional feature representation in the form of a volumetric tensor, where a first dimension corresponds to the temporal progression across the frames, effectively treating time as a “depth” dimension, a second dimension represents the spatial coordinates of the ROI, capturing height and width, and one or more additional feature channels represent iPPG, iBCG, alongside the localized pixel intensity gradients. The prediction unit comprises a hardware-based inference engine interfaced to the signal processing unit and is configured to receive the high-dimensional feature representation in the form of a volumetric tensor from the feature construction module; and apply a second trained neural network model, the second trained neural network model is trained on facial video data and corresponding ground-truth physiological measurements, and is made up of a combination of Convolutional layers, and Transformer with self-attention with positional encoding, specialized in analysing the volumetric tensor, combining temporal, spatial, and physiological feature relationships of the iPPG, iBCG and ROI data of volumetric tensor, to compute at least one physiological metric. The output unit comprises a hardware-based display interface configured to display the predicted at least one physiological metric from the prediction unit, in real-time to a user, providing immediate feedback on the subject's health status. The output unit further comprises a hardware-based probabilistic inference component configured to estimate an uncertainty metric associated with the predicted at least one physiological metric by applying a statistically grounded method, and inference using a trained ensemble of models that provides variance estimates, to yield a quantifiable confidence measure indicating the reliability of the prediction. The communication interface is implemented as a hardware module and is configured to transmit the predicted at least one physiological metric and its associated uncertainty metric to an external device or networked system via a standardized communication protocol; and format the transmitted data following standard healthcare data interchange protocols.

The present disclosure further envisages a method for non-contact, non-invasive health monitoring. The method comprises the steps of capturing real-time digital image data of a subject's face by a camera; receiving the real-time digital image data in a signal processing unit operatively coupled to the camera; detecting facial landmarks using a predefined facial landmark detection algorithm implemented in a video processor; isolating one or more regions of interest (ROIs) based on the detected landmarks to build a real-time series sequence of ROI data, wherein each ROI corresponds to a specific anatomical region; receiving the real-time series sequence of ROI data in a machine learning accelerator; applying a trained neural network model to the real-time series sequence of ROI data to extract Photoplethysmography (iPPG) and Ballistocardiograph (iBCG) signals; preparing features using a feature construction module based on principles of optical coherence tomography (OCT); combining the extracted iPPG and iBCG signals with facial image features derived from the real-time-series sequence of ROI data, wherein the facial image features comprise localized pixel intensity gradients calculated by comparing pixel values within and around the ROI landmarks to capture spatial patterns of intensity variation; constructing a high-dimensional feature representation in the form of a volumetric tensor, where a first dimension corresponds to the temporal progression across the frames, effectively treating time as a “depth” dimension, a second dimension represents the spatial coordinates of the ROI, capturing height and width, and one or more additional feature channels represent iPPG, BCG, and localized pixel intensity gradients; receiving the high-dimensional feature representation in the form of a volumetric tensor in a prediction unit comprising a hardware-based inference engine; applying a second trained neural network model to the high-dimensional feature representation, wherein the second trained neural network model is trained on facial video data and corresponding ground-truth physiological measurements, comprising convolutional layers and Transformer with self-attention and positional encoding, for analyzing the volumetric tensor by combining temporal, spatial, and physiological feature relationships of the iPPG, iBCG, and ROI data to compute at least one physiological metric; displaying the predicted at least one physiological metric on a hardware-based display interface, in real time, to provide immediate feedback on the subject's health status; estimating an uncertainty metric associated with the predicted physiological metric using a hardware-based probabilistic inference component, by applying a statistically grounded method and inference using a trained ensemble of models that provides variance estimates to yield a quantifiable confidence measure indicating the reliability of the prediction; transmitting the predicted at least one physiological metric and its associated uncertainty metric to an external device or networked system via a communication interface implemented as a hardware module; and formatting the transmitted data according to standard healthcare data interchange protocols.

In an embodiment, the sequence of frames is recorder under controlled lighting conditions or dynamically adjusted exposure settings to ensure consistent pixel intensity values.

In an embodiment, the predefined facial landmark detection algorithm is selected from a group consisting of, but not limited to: a Haar cascade classifier, and a deep learning based facial landmark model, stored in on-chip memory, the algorithm identifying reference points such as corners of the eyes, edges of the nostrils, and corners of the mouth.

In an embodiment, the specific anatomical region consisting of, but not limited to: the cheeks, forehead, and the nose, which are known to exhibit minute pixel intensity fluctuations correlated to blood perfusion and micro-movements induced by the cardiovascular and respiratory activity.

In an embodiment, the invention is used to detect nervousness, rapid blinking, wide eyes, and other signs associated with smugglers or terrorists. The classic polygraph measures blood pressure, pulse, breathing rate and sweating as well as in some cases movement indicating stress that can signify deception. Apart from sweating, these measures can be captured remotely and non-invasively by this invention, potentially allowing for individual and crowd monitoring in transportation settings (train stations, airports), business settings (banks, office buildings) and law enforcement settings (suspect interviews). A real-world applicable example is security checkpoints at airports, which currently have a very difficult job. They need to spot people intent on criminal behavior without disrupting the flow of passengers. Currently the TSA relies on data compiled on individual travelers, data related to the traveler's country of origin, and on personal observation of common actions associated with people who are nervous about being screened. The invention, when installed in a TSA checkpoint, can rapidly identity likely-criminal intentions by analysing the physiological signature embedded in a short facial video clip. This makes the TSA's ability to stop smugglers and terrorists easier, quicker, and more comprehensive.

In an embodiment directed to law-enforcement and investigative use, the invention, non-contact vital-sign inference provides a passive complement to conventional polygraph methodologies. Without electrodes or direct skin contact, optical cameras are used to analyze micro-variations in breathing, cardiovascular signals and stress-related markers as proxies for autonomic arousal. During interviews or interrogations, the system can furnish real-time indications of stress dynamics and response patterns without interrupting the natural flow of questioning, while portable field configurations permit immediate assessment in less controlled environments.

In an embodiment, the invention includes a dash camera in vehicles or remote patient bedside monitoring. For example, during a 2½ year period between July 2005 and December 2007, the National Highway Transportation Authority estimated that nearly 50,000 drivers had medical emergencies that led to vehicle accidents. How many of those accidents could have been prevented if this invention was installed in cars to allow a warning to be sent to the driver to get off the road BEFORE the medical emergency? That is a question that can only be answered once this invention becomes a mainstay in all vehicles. Just being able to assess a driver's heart rate in real time provides an excellent predictive tool for cardiac arrests, seizures, and other maladies. The current invention allows for this, and other assessments of possible dangers such as alcohol and drug impairment, drowsiness and distraction, in real time, allowing either the driver or a person outside of the vehicle to warn of a possible impending medical emergency.

In an embodiment, the system continuously monitors the driver's heart rate, respiration and heart-rate variability using the driver-facing camera, without requiring any wearable device, to maintain a live appraisal of the driver's state. When the signals indicate emerging fatigue or drowsiness, the system issues timely alerts to avert incidents; when they indicate heightened stress or cognitive load during demanding driving, the vehicle may adapt cruise control, brake sensitivity and steering response to stabilize performance. By correlating these physiological measures with vehicle telemetry, the system further supports predictive maintenance by identifying patterns in which driver behavior and physiological state are associated with mechanical strain, thereby providing a dual safety layer that safeguards the human operator and reinforces mechanical reliability.

From a legal perspective, car and truck manufacturers are increasingly adding driver-facing cameras to new vehicles in anticipation of the establishment of a national safety standard for passive, advanced impaired driving prevention technology by the National Highway Traffic Safety Administration (NHTSA) which is expected in 2026. Using these cameras for their intended purpose—accident prevention by recognizing driver impairment early—is an embodiment of the proposed technology.

Turning to the invention at hand, video of a driver's face is analyzed in real time detecting variations in heart rate, heart rate variability (HRV), blood pressure and blood oxygen concentration. Changes in each of these indicators, individually, can predict a potential health emergency, and several factors have been shown to be particularly dangerous, warranting a warning to a driver to stop the vehicle and seek help:
A hypertensive crisis, meaning an episode of exceptionally high blood pressure (180/120 mm HG) is a medical emergency and can precipitate a stroke. High blood pressure is known as the “silent killer” because even severe spikes in blood pressure are often not felt or noticed by a patient. If warned by an in-car safety system to stop, such as that provided by this invention, a driver could see immediate help and avoid a potentially fatal accident.
Heart Rate Variability—whether measured by a wearable heart monitor or PPG has been shown to be a reliable predictor of epileptic seizure, allowing drivers with epilepsy critical minutes or seconds to assure their own safety during the seizure, and thereby also avoiding potentially deadly accidents. According to the National Motor Vehicle Crash Causation Survey (NMVCCS) statistics on crashes precipitated by drivers' medical emergencies an estimated 8750 accidents per year are due to a driver seizure. Non-invasive driver heart rate variability measurement to predict and give an early warning about an oncoming seizure could make these accidents a thing of the past. Likewise, an ECG (electrocardiogram) is the standard to diagnose a heart attack. In-car driver monitor technology informing a driver that they're experiencing a heart attack and must stop the vehicle immediately could save both the driver and many around them. Thus, the invention can not only allow drivers facing a potentially life-threatening medical condition to save themselves, but also the other drivers on the road.
Non-invasive, non-intrusive use of the increasingly common driver-facing cameras in cars and commercial trucks in combination with the Applicant's technology allows for passive driver health monitoring and warning if the driver is (about to) experiencing a seizure, heart attack or stroke.

Of particular importance in this embodiment is the use of the invention to monitor in real time the physical well-being of truck drivers by their companies. Not a week goes by without another tragic story of a commercial trucker who became incapacitated and died at the wheel, and/or killed some other drivers/pedestrians. Just during the week that this patent application was being prepared, a trucker suffered a heart attack and killed five children in a church van. Because driving a truck is a very sedentary job, it is estimated that over 40% of commercial truck drivers have a high risk of cardiovascular disease. It is also well known in the industry that some truckers ignore the safely rules regarding the length of time they need to rest in between legs of driving, and other use amphetamines and other drugs to stay awake. Currently, trucking companies look at their truckers' logs when they return from a trip, and ask them to call in to report where they are, but this is inadequate, as can be seen from the number of trucker-related fatalities related to either a lack of sleep or a medical emergency.

In an embodiment, the at least one physiological metric is including: pulse rate, breathing rate, and blood oxygen saturation (SpO2), blood pressure, and heart rate variability.

In an embodiment, the statistically grounded method is selected from a group consisting of, but not limited to: Bayesian inference using a prior and likelihood model.

In an embodiment, the standardized communication protocol is selected from a group consisting of, but not limited to: Wi-Fi, Bluetooth, or Ethernet.

In an embodiment, the standard healthcare data interchange protocols are selected from a group consisting of, but not limited to: HL7, FHIR protocols, ensuring compatibility with electronic health record systems or cloud-based analytics platforms.

In an embodiment, the invention can detect spoofing. Liveness of a person pertains to instances of virtually impersonating another person or veiling one's identity by manipulating the physical appearance of the individual during a video-meeting or video-call. High-profile fraud cases of people purporting to be someone else have emerged in job interviews as well large, fraudulent financial transactions. A real-time measure of liveness of an individual adds trust to a transaction. Several years ago, a series of humorous videos emerged with current president Trump having casual conversations with former presidents Biden and Obama about silly things like the best flavor of ice cream, the best video games, etc. Today, artificial intelligence, and the abuse of it are no longer a funny game. In 2024 a Hong Kong CFO was spoofed as endorsing products, resulting in a $25 million dollar financial scam. Actors such as Scarlett Johansson are actively critical of AI attempts to mimic their voice and in some cases, their bodies and movement, there is an active industry is using AI to resurrect dead actors and AI is being used to create “digital doubles” of famous actors to perform stunts. It is of concern to actors around the world whether they will still have jobs once the digital doubles are realistic enough to handle an entire movie roll.

Spoofing is also becoming ingrained in academia. Cybercriminals are increasingly singling out researchers, alongside politicians and celebrities. For example, Kgomotso Mathabe, a respective urologist In South Africa, was recently shocked to find that a spoof of her was promoting an erectile dysfunction drug In deepfake videos on social media. The video of Mathabe was a deepfake, generated using artificial intelligence (AI) technology trained on real video and audio material. Such videos have become difficult to distinguish from the real thing, as well as easier and cheaper to make, so their harmful use is a growing concern. In this particular case, viewers of the deepfake video were directed to a scam website which asked them to enter their banking details “to get the drug shipped to them”. Those who did so had money siphoned out of their account, often several times, and received no “medicine” in return.

Scientific spoofing is a world-wide problem. In India, diabetes specialist Viswanathan Mohan has been featured in several deepfake videos, including one in which he seems to be talking in Hindi, a language that he doesn't speak. Because Mohan Is one of the foremost diabetes scientists In India, deepfake videos of him promoting scam products damage both the unwitting people who buy the products, but also Mohan's previously-sterling reputation as well. Rather than doing damage control on the financial losses and the scientist's reputation, wouldn't It be easier to use the current Invention to detect spoofing before the Internet platform releases the video?

Fake job applicants are another major problem that could be solved by the current invention. It is estimated that the cost of finding and hiring an employee is approximately 30% of their salary. Spoofing-technology allows users to alter their physical appearance, the sound of their voices and the content of their speech in video calls have allowed scammers—often from other parts of the world—access to highly paid jobs and sensitive data. Beyond the defrauded US employer, there is a broader effect on the US job market, making it harder for qualified Americans to land well-paying jobs. A recent example, reported by the US Department of Justice includes a group of 14 North Korean nationals working in remote jobs in 300 US companies under false identities for years, defrauding US companies for over $88M. Assessing the vital signs, as provided by this invention, to detect “real-ness” of a person during a virtual interview (on Zoom, MS Teams, etc.) would allow employers to weed out fake applicants during the interview process. Since it is becoming more and more obvious that we can no longer trust our eyes to ascertain that a person on a call is real, technology testing for real-ness buys peace of mind.

The device is equipped to operate across various platforms, including mobile devices, desktops, and cloud-based servers, ensuring wide compatibility. Privacy protection is emphasized through on-device data processing and an optional federated learning approach, where model updates are performed without compromising user privacy. By processing data locally, sensitive health information remains on the user's device, meeting stringent privacy standards such as GDPR (General Data Protection Regulations) and HIPAA (Health Insurance Portability and Accountability Act).

Motor-vehicle collisions precipitated by sudden driver medical events remain a significant public-safety concern; national crash-causation surveys attribute on the order of twenty-thousand incidents per year—approximately 1% of all recorded crashes—to driver seizure, cardiac infarction, or similar health emergencies. In one widely reported 2025 incident, a driver experiencing a seizure veered into an outdoor daycare facility, resulting in multiple fatalities. In a separate tractor-trailer crash, a driver suffering an acute myocardial infarction caused a multivehicle pile-up that claimed five children. Commercial-driver studies further show that more than forty per-cent of operators are at elevated cardiovascular-disease risk. These statistics underscore the value of a contact-free, in-cabin monitoring subsystem capable of detecting pre-symptomatic hemodynamic anomalies and issuing an early warning.

Prior “remote PPG” (rPPG) solutions relying solely on green-channel intensity analysis and classical digital-signal-processing pipelines cannot meet the latency (<100 ms) and motion-tolerance (>±30 deg head rotation; 10 lux-1 000 lux illuminance change) demanded by automotive and heavy-equipment environments. Nor can they distinguish live subjects from presentation attacks with sufficient confidence for high-assurance security applications.

Advantages of the Disclosed OCT-Inspired Fusion Network. By fusing volumetric iPPG and kinematic iBCG feature maps in an optical-coherence-tomography-style neural architecture, the present device delivers stable vital-sign estimates from one-second video windows at frame rates of 30 fps, with error rates below five per-cent against medical-grade references. The same architecture simultaneously computes a liveness score: authentic videos exhibit quasi-periodic, cross-correlated iPPG-iBCG signatures, whereas replay or AI-generated content does not. Consequently, the system supports:
a. advanced driver-assistance modules that trigger vehicle intervention or driver alerts upon detection of hypertensive crisis, pre-seizure heart-rate-variability patterns, or myocardial-ischaemia indicators;
b. operator-safety monitoring for cranes, haul-trucks, and construction machinery, where real-time hemodynamic surveillance mitigates fatigue-related accidents;
c. security checkpoints or remote-hiring platforms that verify subject liveness and thwart deep-fake or spoof attempts by analysing the physiological signature embedded in a short facial video clip.
Accordingly, the disclosed non-contact health-monitoring system-uniquely enabled by OCT-inspired deep-feature fusion of iPPG and iBCG signals-addresses long-felt needs in transportation safety, industrial operations, tele-healthcare, and digital-identity verification, domains in which prior rPPG-only or conventional DSP approaches have proven inadequate.

Other and further aspects and features of the disclosure will be evident from reading the following detailed description of the embodiments, which are intended to illustrate, not limit, the present disclosure.

### BRIEF DESCRIPTION OF DRAWINGS

The illustrated embodiments of the subject matter will be best understood by reference to the drawings, wherein like parts are designated by like numerals throughout. The following description is intended only by way of example, and simply illustrates certain selected embodiments of devices and methods that are consistent with the subject matter as claimed herein, wherein:

FIG. 1 illustrates an overall architecture of the non-contact health monitoring device, including key components such as the camera, signal processing unit, prediction unit, and output modules, in accordance with an embodiment of the present disclosure;

FIG. 2 illustrates a data flow diagram showing the process of capturing facial video data, extracting physiological signals, and predicting health metrics through machine learning models, in accordance with an embodiment of the present disclosure;

FIG. 3 illustrates mathematical framework and algorithms applied to derive physiological metrics from the high-dimensional feature tensor constructed by the processing unit, in accordance with an embodiment of the present disclosure;

FIG. 4 illustrates the internal operations of the feature construction module, detailing the integration of iPPG and iBCG signals with pixel intensity gradients, in accordance with an embodiment of the present disclosure;

FIG. 5 illustrates the device integrated with an external monitoring application, highlighting data communication and alert functionalities, in accordance with an embodiment of the present disclosure;

FIG. 6 illustrates the device embedded in automotive setups, such as driver monitoring systems, for real-time health and alert notifications, in accordance with an embodiment of the present disclosure;

FIG. 7 illustrates the integration of the device within fitness equipment for monitoring health metrics during exercise, in accordance with an embodiment of the present disclosure;

FIG. 8 illustrates the device embedded in consumer electronics like televisions, offering wellness monitoring in home environments, in accordance with an embodiment of the present disclosure;

FIG. 9 illustrates the application of the device in telehealth, enabling real-time remote health assessments during virtual consultations, in accordance with an embodiment of the present disclosure; and

FIGS. 10A-10C illustrate a method for non-contact, non-invasive health monitoring in accordance with an embodiment of the present disclosure.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION

A few inventive aspects of the disclosed embodiments are explained in detail below with reference to the various figures. Embodiments are described to illustrate the disclosed subject matter, not to limit its scope, which is defined by the claims. Those of ordinary skill in the art will recognize a number of equivalent variations of the various features provided in the description that follows.

### Non-Limiting Definitions

Definitions of one or more terms that will be used in this disclosure are described below without limitations. For a person skilled in the art, it is understood that the definitions are provided just for the sake of clarity and are intended to include more examples than just provided below.
Camera: A device configured to capture real-time image data of a user's face, comprising a sequence of frames recorded under controlled lighting or dynamically adjusted exposure settings to ensure consistent pixel intensity values.
Signal Processing Unit: A hardware component operatively coupled to the camera, responsible for receiving and analyzing image frames. It includes:
Video Processor: Dedicated circuitry designed to detect facial landmarks and isolate specific Regions of Interest (ROIs).
Machine Learning Accelerator: Specialized circuitry that extracts physiological signals such as Photoplethysmography (iPPG) and Ballistocardiography (iBCG) using trained neural network models.
Facial Landmark Detection Algorithm: A predefined computational method, such as Haar cascade classifiers or deep learning models, for identifying reference points on the face including eyes, nostrils, and mouth corners.
Regions of Interest (ROIs): Anatomical regions of the face (e.g., cheeks, forehead, nose) exhibiting physiological variations, identified based on detected facial landmarks.
Photoplethysmography (iPPG): Represents blood volume changes detected via pixel intensity variations.
Ballistocardiography (iBCG): Reflects micro-motions induced by cardiac activity through facial tissue displacements.
Feature Construction Module: Hardware logic implementing optical coherence tomography (OCT) principles to combine iPPG and iBCG signals with facial image features into a high-dimensional feature representation, reducing noise and improving stability.
Volumetric Tensor: A multi-dimensional data structure containing temporal progression of image frames, spatial coordinates of ROIs (height and width), and additional feature channels for iPPG, iBCG, and pixel intensity gradients.
Prediction Unit: A hardware-based inference engine configured to process volumetric tensors using advanced machine learning models (e.g., CNNs, Transformers) to compute at least one physiological metric.
Output Unit: A display interface providing real-time feedback of physiological metrics.
Probabilistic Inference Component: Provides uncertainty metrics for the predictions using Bayesian inference techniques.
Communication Interface: A hardware module transmitting physiological metrics and uncertainty data to external devices via standardized protocols (e.g., HL7, FHIR, Wi-Fi, Bluetooth).
Physiological Metric: Quantifiable health parameters such as heart rate, respiratory rate, blood pressure, and oxygen saturation, computed with error rates within medically validated criteria.
Standardized Communication Protocols: Protocols ensuring data transmission compatibility, including Wi-Fi, Bluetooth, Ethernet, and healthcare-specific standards like HL7 and FHIR.
Healthcare Data Interchange Standards: Structured frameworks for formatting transmitted health data to ensure integration with electronic health record systems or cloud-based analytics platforms.

### Overview

Traditional health monitoring methods rely on wearable devices or physical contact sensors, which may cause discomfort, restrict movement, and limit usage in certain environments such as public spaces or within vehicles. Additionally, these methods can be impractical or inaccessible in remote areas or situations requiring quick, large-scale health assessments, such as during pandemics or mass gatherings. The challenge lies in achieving reliable, real-time health insights that are convenient, accessible, and suitable for a wide range of users without compromising accuracy.

To solve this problem, the present disclosure introduces an AI-driven device that utilizes high-resolution facial video data to analyze physiological signals and estimate a range of vital signs and wellness parameters. Using a camera to capture facial data, the device processes this data through facial landmark detection and deep learning models to detect Photoplethysmography (iPPG) and Ballistocardiograph (iBCG) variations in specific regions of interest. These physiological variations are processed into a high-dimensional feature vector and input into a model combining Convolutional Neural Networks (CNNs) for spatial analysis and Transformer-based attention mechanisms for temporal pattern recognition. This approach allows the device to predict vital signs such as heart rate, blood oxygen levels, and blood pressure, as well as wellness indicators like stress and immune health, without requiring any physical interaction with the user.

The disclosed system advances non-contact vital-sign estimation by enabling liveness verification and spoof detection from a video interval as short as one second captured at a frame rate of at least thirty frames per second. In genuine footage, imaging photoplethysmography (iPPG) and imaging ballistocardiography (iBCG) signals extracted from a facial region of interest are quasi-periodic and mutually correlated with the underlying heartbeat; in replay attacks or AI-generated deep-fake content, those signals are absent or manifest as temporally incoherent noise, and principal-component energy fails to align with a physiological cadence. The system therefore: (i) acquires iPPG and iBCG feature maps; (ii) concatenates the feature maps into a volumetric tensor by means of an optical-coherence-tomography (OCT)-inspired deep-feature-fusion network; and (iii) classifies the tensor in real time to determine liveness. The fusion architecture preserves both hemodynamic amplitude and kinematic phase information, permitting inference latencies below fifty milliseconds and delivering accurate spoof-detection performance for one-second video clips in dynamic, real-world environments.

### System Overview

In one embodiment, the invention provides a non-contact, non-invasive health monitoring device that can measure both cardiopulmonary vital signs and metabolic biomarkers using only an RGB video of a user's face. This device is built around a camera-based sensing architecture coupled with advanced signal processing and deep learning models. The camera captures real-time facial image data under controlled illumination conditions (e.g. constant artificial lighting of approximately 300-500 lux) to ensure consistent pixel intensity values. The system then analyses subtle variations in the facial video frames to extract physiological signals and predict health-related metrics. An overview of the device's operation involves:
a. Facial ROI Extraction: Detecting the user's face and identifying key regions of interest (ROIs) (such as the forehead, cheeks, and nose) that are most indicative of blood flow and micro-motions from cardiac activity.
b. Physiological Signal Derivation: Using a trained neural network to extract imaging photoplethysmography (iPPG) and imaging ballistocardiography (iBCG) signals from the ROI video data, capturing subtle pulse-induced color changes and heart-induced micro-movements in the face.
c. Feature Tensor Construction: Combining the extracted signals with spatial facial features to form a high-dimensional volumetric tensor that encodes temporal, spatial, and physiological information from the video.
d. Health Metric Prediction: Feeding the feature tensor into a second deep learning model that estimates one or more physiological metrics (e.g. heart rate, blood pressure, blood oxygen levels, and metabolic biomarker levels like glucose) for the user.
e. Output and Feedback: Displaying the predicted health metrics to the user in real-time and optionally transmitting the data to external devices or health record systems, along with a confidence estimate indicating prediction reliability.

### Dataset Collection and Preparation

To train the system's models and validate its accuracy, a comprehensive multi-modal dataset of facial videos with corresponding ground-truth physiological measurements was collected. This dataset spans hundreds of participants of varying ages, sexes, and skin tones, ensuring the resulting models generalize across demographics. Each participant was recorded in multiple sessions to capture a broad range of physiological states and conditions:
a. Rest and Active States: Videos were taken during baseline resting periods (e.g. seated calm, morning fasted state) and after induced stressors like exercise. For example, a subject might be recorded at rest and then again immediately after performing physical activity (such as a set of squats) to induce changes in heart rate and circulation.
b. Diurnal Variations: Sessions were scheduled at different times of day (morning, afternoon, evening) to incorporate natural variations in vital signs and metabolic state that occur over a day.
c. Pre- and Post-Meal: Participants were recorded in a fasting state and then again after consuming a meal, to capture how cardiovascular signals and metabolic biomarkers (like glucose and triglycerides) change from pre- to postprandial states.

During each video recording session, medical-grade instruments provided synchronized ground-truth measurements of vital signs and metabolic biomarkers:
a. Heart Rate, Respiratory Rate, and HRV—Continuously measured using contact sensors such as finger photoplethysmography (BVP sensor) and electrocardiogram (ECG) leads. Heart rate variability (HRV) metrics were later derived from the ECG/BVP signals by calculating the variations in time intervals between successive heartbeats.
b. Blood Pressure—Systolic and diastolic pressure captured at intervals using an oscillometric blood pressure cuff.
c. Blood Oxygen Saturation (SpO₂)—Monitored with a fingertip pulse oximeter throughout the session.
d. Metabolic Biomarkers—At specific points (e.g. before and after a meal), finger-prick blood samples were taken and analyzed for glucose level, total cholesterol, ketone bodies, and uric acid. This was done with standard lab equipment and in some cases the portable point-of-care devices (such as EasyTouch® analyzers), providing ground-truth values for the model to learn from.

All video and sensor data were time-synchronized using visible timing cues (for example, an LED timer or digital clock visible in the video frame) to align each video frame with the exact moment of each sensor reading. Data collection followed strict ethical protocols: participants gave informed consent for their facial videos and medical data to be used in the study. To ensure data quality, recordings were conducted in a controlled environment. Subjects sat facing the camera with consistent framing, and lighting was kept uniform (while still allowing some variation across sessions to improve robustness). Environmental factors like background and camera model were also varied slightly across sessions so that the models would not overfit to a single setting.

The raw video frames were then passed through a series of preprocessing steps to maximize the physiological signal-to-noise ratio:
a. A face-detection and landmark localization algorithm (e.g. a MediaPipe Face Mesh model) automatically identified the boundaries of the face and key facial landmarks on each frame. This allowed the system to define consistent ROIs (such as the center of the forehead, the high cheek areas under each eye, and the nose bridge), which are known to exhibit strong pulsatile signals due to dense blood flow.
b. The identified ROIs were tracked across frames so that if the subject moved slightly, the ROI windows would follow along based on the facial landmarks. This motion compensation ensured that the same skin regions were analyzed over time despite minor head movements, preventing spurious signal jumps.
c. A skin mask was applied to each frame to exclude background and non-skin pixels. By zeroing out or de-weighting all non-skin regions, the system focused only on the subject's skin, eliminating noise from irrelevant background motion or lighting changes in the environment.
d. Illumination normalization was performed to adjust for any global lighting variation over time. For instance, a rolling average of the pixel intensity in each ROI could be computed, and each frame's ROI pixels could be normalized (through subtraction or division) by this average to remove slow drifts in brightness (analogous to removing a DC offset from a signal).
e. Each ROI's color signal was further normalized to account for differences in skin tone and camera sensor response. For example, the mean pixel value of an ROI (over a time window or the whole video) could be removed so that the analysis concentrates on temporal fluctuations rather than static color. This step prevents biases in the model due to a person's skin color or the camera's exposure settings.
f. Spatial downsampling of each ROI (for example, combining blocks of pixels into “super-pixels”) was done to reduce high-frequency sensor noise and decrease data dimensionality. This preserves the overall waveform of color changes (containing physiological signals) while smoothing out pixel-level noise.

As a result of this preprocessing pipeline, each video session was converted into a set of cleaned, normalized ROI time-series, ready for feature extraction. The combination of stable face tracking, skin masking, and brightness normalization greatly improved the quality of the extracted signals by removing irrelevant fluctuations and emphasizing true physiological changes in the skin's appearance.

In addition to the supervised collected participant data, the training process also leveraged existing public video databases to increase the variety of facial inputs. For instance, videos from open-source activity recognition datasets were repurposed. Because these external videos lacked direct physiological measurements, digital signal processing techniques were employed to derive synthetic iPPG and iBCG “labels” from them. In other words, algorithmic estimates of the pulse waveform and cardiac micro-motions were computed from these videos offline, and those estimated signals served as pseudo-ground-truth for pre-training the iPPG/iBCG extraction model. By training on both the real data (with accurate measured vitals) and the augmented data (with synthetic labels), the system's neural networks learned to extract robust physiological features from video even under varied conditions.

Finally, the prepared dataset was structured for supervised learning. Each video recording was divided into short segments (or “chunks”) of consecutive frames. Each chunk was treated as a training sample: the sequence of normalized ROI frames (and associated skin masks) was the input, and the corresponding set of physiological measurements (averaged or interpolated over that time window) was the label. For example, a 30-second video chunk might be paired with the average heart rate, respiratory rate, and blood glucose level measured during that interval. Organizing the data into these aligned chunks allowed the models to learn both short-term patterns (within a chunk) and how those relate to the quantitative health metrics at that time. This approach facilitated efficient training of the deep learning models for both vital signs and metabolic biomarker prediction.

### Methodology

The device's signal processing unit receives the live video stream from the camera and converts it into the key physiological signals and features needed for health metric prediction. This unit is composed of dedicated hardware modules for real-time performance:
a. Video Processor (Facial ROI Module): A specialized video processing circuit runs a facial landmark detection algorithm (for example, a Haar cascade classifier or a deep neural network model stored in on-chip memory) to identify the face and locate key facial landmarks in each frame. It quickly finds reference points such as the corners of the eyes, the edges of the nostrils, and the corners of the mouth. Using these landmarks, the processor then isolates predetermined ROIs on the face (for instance, an ROI spanning the forehead region, one on each cheek, and one covering the nose bridge area). These regions are chosen because they are rich in blood perfusion and also exhibit subtle motions corresponding to cardiac and respiratory activity. The output of this module is essentially a set of ROI image streams (or the coordinates of those ROIs over time) extracted from the full frame, which will be further analyzed for physiological signals.
b. Machine Learning Accelerator (iPPG & iBCG Extraction): This specialized integrated circuit executes a trained neural network model to derive the iPPG and iBCG signals from the incoming ROI image sequences. The neural network architecture employed here includes transformer blocks with self-attention and positional encoding, enabling it to capture complex spatio-temporal patterns in the video data. When fed with a time-series of ROI frames, the model analyzes tiny fluctuations in pixel intensity and minute movements of facial tissue that occur with each heartbeat and breath. From these subtle patterns, it outputs features corresponding to the two waveforms: the iPPG (imaging photoplethysmogram), which reflects blood volume changes (pulse) in the facial microvasculature, and the iBCG (imaging ballistocardiogram), which reflects the slight mechanical movements of the head or face due to the force of cardiac ejection and respiration. This iPPG/iBCG extraction model was trained in advance using video data synchronized with ground-truth signals (such as contact PPG, ECG, or seismocardiography), and improved with semi-supervised learning techniques to make use of unlabeled video segments. As a result, the accelerator can reliably compute pulse and micro-motion signals from the face in real time, even in the presence of minor motion or noise.
c. OCT-Inspired Feature Construction Module: After obtaining the primary physiological waveforms (iPPG and iBCG), the signal processing unit combines these with additional context from the video in a feature construction stage. Implemented in hardware logic, this module takes the iPPG and iBCG signals and fuses them with localized facial image features to create a rich, volumetric feature tensor. One key set of features comes from computing localized pixel intensity gradients around the ROIs and landmarks—essentially capturing how pixel values change across small spatial neighborhoods on the face. (For example, this can highlight patterns like the gradient of redness across the cheeks or the edges of pulsatile areas.) The module then constructs a multi-dimensional tensor where: two dimensions correspond to spatial coordinates (height and width across the assembled ROIs or a facial grid), one dimension corresponds to time (the sequence of frames, treated analogously to a “depth” dimension as in a 3D image), and additional dimensions or channels encode different feature types (raw normalized pixel values, computed gradient maps, and temporal signals like the iPPG and iBCG). This process is inspired by optical coherence tomography (OCT) in the sense that it compiles a stack of cross-sectional data (here, temporal cross-sections of facial image and signal data) into a volume for analysis. The outcome is a high-dimensional representation that preserves the critical information needed to predict health metrics, while filtering out irrelevant noise. By performing operations such as background subtraction, motion compensation, and multi-channel fusion at this stage, the feature construction module ensures the subsequent prediction model receives input data that is de-noised and stabilized against artifacts.

The prediction unit receives the feature tensor from the signal processing unit and runs a second-stage deep neural network to estimate the user's physiological metrics. This unit is realized in hardware as a neural network inference engine, optimized to handle the model's computations with minimal latency for real-time results. The neural network architecture is designed to jointly analyze the spatial, temporal, and physiological information encoded in the volumetric tensor. For example, initial layers of the network are convolutional, scanning through the tensor to detect higher-level features (such as pulse wave patterns or motion trends) in both space and time. These are then followed by transformer layers with self-attention, which allow the model to dynamically focus on particular time points or ROI regions that are most informative for a given prediction. Positional encoding in the transformer ensures that the model keeps track of the order of frames and the layout of ROIs while making sense of the temporal dynamics. To exploit complementary mechanics of iPPG and iBCG, a phase-coherence regularizer maximizes the magnitude-squared coherence between the iPPG fundamental and the iBCG J-wave envelope, encouraging physiologically plausible outputs under motion. This is implemented as a differentiable spectral loss added during training. This second neural network is trained on the synchronized facial video and ground-truth measurement data described earlier. It learns to map patterns in the feature tensor to specific physiological outputs. In the primary configuration, the model is trained to predict key cardiopulmonary vital signs such as:
a. Heart Rate (HR)—The pulse rate in beats per minute, derived from the periodic patterns in the iPPG signal (e.g. detecting peaks corresponding to heartbeats).
b. Respiratory Rate (RR)—The breathing rate in breaths per minute, inferred from slower modulations in the signals or subtle cyclical motions of facial features (for instance, slight head bobbing or nostril dilation with breathing).
c. Heart Rate Variability (HRV)—Metrics quantifying variations in heartbeat intervals (such as the standard deviation of inter-beat intervals), which the model can derive by analyzing the consistency of the iPPG pulse-to-pulse timing.
d. Blood Oxygen Saturation (SPO₂)—An estimate of blood oxygen levels. While the system uses a normal RGB camera (not a dedicated pulse-oximeter with red/infrared light), the model can infer SpO₂ from learned correlations in the facial color changes and iPPG waveform shape, especially if trained on data where SpO₂ varied. For example, certain changes in the amplitude ratio of the pulse waveform under different color channels might carry SpO₂ information.
e. Blood Pressure—Systolic and diastolic blood pressure, estimated by recognizing patterns in the combined iPPG and iBCG signals that correlate with blood pressure dynamics. For instance, the model may learn relationships akin to pulse transit time (the delay between electrical cardiac events and peripheral pulse) or the intensity of the BCG movement, which have known correlations with blood pressure.

Through extensive training on many individuals and conditions, the prediction model can output these vital signs with high accuracy (in testing, errors were consistently under 5% when compared to clinical instrument readings, meeting standard medical accuracy criteria). The model effectively personalizes its predictions on the fly using the input data features, without needing explicit calibration for each new user, thanks to the robust training and normalization.

The device provides immediate feedback via an output unit once the physiological metrics are computed. This includes a user interface (for example, an on-device display or a connected smartphone app) that presents the measured vital signs and other health indicators in real time. Each displayed metric can be accompanied by an uncertainty estimate or confidence level. For example, the system might show a heart rate reading as “75±3 bpm,” indicating a confidence interval around the prediction. This uncertainty quantification is generated by a probabilistic inference component of the output unit. In practice, the system uses a Bayesian neural network in the background; if all models agree closely on a value, the uncertainty is small, whereas if there is disparity (perhaps due to poor signal quality or motion), the uncertainty range widens. Providing this kind of feedback helps the user (or a clinician) gauge the reliability of the readings at a glance.

The prediction network is trained in a multitask regime with per-output heteroscedastic uncertainty heads. Let y^{k} be the ground-truth metric k (e.g., HR, RR, SBP, DBP, SpO₂, glucose, ketones), μ^{k}, σ^{k} the network's mean and log-variance outputs. The loss sums task-wise negative log-likelihoods L=Σₖ ((y^{k}−μ^{k})²·exp(−σ^{k})+σ^{k}), which (a) balances tasks without manual weighting and (b) yields calibrated confidence intervals shown to users. A signal-quality index (SQI), computed from the iPPG/iBCG peak-to-noise ratio and spectral coherence at 0.7-3 Hz, gates inference; windows with SQI below a threshold are flagged as low-confidence.

In addition to raw numbers, the output interface includes trend graphs or indicators (e.g. an increasing or decreasing arrow next to a metric to show its recent trajectory). It also issues alerts or recommendations if a metric is outside of a healthy range or shows an abrupt change. For instance, if the detected blood pressure is very high, the system could display a warning or advise the user to rest and measure again. The design, however, focuses on monitoring and early alerts rather than making definitive medical diagnoses; users are encouraged to seek professional confirmation for any alarming readings.

The device is equipped with a communication interface that enables it to transmit the measured data and insights to external systems securely. This module supports common communication protocols—for example, wireless connectivity via Wi-Fi or Bluetooth for syncing with mobile devices and networks, and optional wired connections like Ethernet or USB for direct interface with computers or docking stations. Data is packaged and transmitted following standard healthcare data interchange formats, such as HL7 or FHIR (Fast Healthcare Interoperability Resources), to ensure compatibility with electronic health record systems and other health IT platforms. In practice, a user could configure the device to automatically send their daily measurements to a cloud database or a healthcare provider. Each transmission is protected with encryption and proper authentication to maintain privacy and data security, as the information constitutes sensitive personal health data. This connectivity and adherence to standards make it easy to integrate the device into telemedicine workflows or personal health tracking regimens, allowing the facial-video-derived health metrics to complement traditional medical data.

One important extension of this system, in certain embodiments, is its ability to estimate metabolic biomarker levels (such as blood glucose, total cholesterol, ketone bodies, and uric acid) from the same facial video data, without any invasive measurements. This is made possible by leveraging the rich information in the volumetric feature tensor and training the deep learning models to recognize patterns associated with metabolic changes. In this extended mode of operation, the prediction model is configured as a multi-task network—it not only outputs the cardiopulmonary vitals described above, but also additional outputs corresponding to these metabolic indicators.

Training for this extended capability involves incorporating the metabolic ground-truth data into the model's learning process. As described in the dataset collection, each video session is paired with measurements of glucose, lipids, ketones, etc., particularly capturing variations such as fasting versus post-meal states. The neural network learns subtle correlations between the facial signals and these metabolic states. For instance, changes in blood perfusion dynamics or heart rate patterns after a meal may correlate with rising glucose levels; similarly, systemic effects of elevated cholesterol or the presence of ketone bodies (e.g. during ketosis) impart slight changes in the iPPG and iBCG waveforms. By exposing the model to many examples of these scenarios across different individuals, it gains the ability to predict an individual's metabolic biomarker levels from their video with a useful degree of accuracy.

For these metabolic biomarkers, labeling follows a fixed protocol: venous draw (lab analyzer) or finger-prick (IFCC-traceable point-of-care device), units (mg/dL for glucose/uric acid, mg/dL for total cholesterol, mmol/L for β-hydroxybutyrate), sampled at: T₀ (≥8 h fast), T₁ (30-60 min post-meal), T₂ (90-120 min post-meal), and optional post-activity timepoints. Training targets are either continuous values (standardized by subject-agnostic population stats) or ordinal bands (e.g., normal/elevated/high). Performance is reported as MAE and % within clinically accepted grids (e.g., Clarke grid zones A+B≥95% for glucose trend classification). The network does not diagnose disease and is intended for trend guidance with uncertainty bounds.

It is noteworthy that these predictions are made in a non-personalized manner—the model does not require any prior calibration to a specific user's baseline values. Thanks to the diverse training data and the normalization steps (which remove static biases like absolute skin color or lighting differences), the system can generalize to new users and provide estimates of metabolic metrics that reflect deviations from normal ranges. Of course, predicting blood chemistry from video alone is an emerging and challenging task, so the system's estimates (for example, an estimated blood glucose value in mg/dL) are presented alongside an uncertainty range and are intended to guide or alert the user rather than serve as a definitive diagnostic. In evaluations on the collected dataset, the device's glucose trend predictions were able to distinguish high vs. normal blood sugar levels with high sensitivity, and similarly, its cholesterol and ketone level indications correlated with the actual measured values well enough to flag significant elevations or changes.

In practice, a user could enable this extended feature by performing a standard recording session (e.g. a 30-second facial scan under the same controlled conditions). The device would then output not only vital signs like heart rate and blood pressure, but also an estimate of key metabolic health metrics like current glucose level or whether the user's ketone level suggests fat-burning metabolism. This provides a more holistic health snapshot from a simple video, combining cardiovascular and metabolic information. Crucially, this extension requires no change in hardware—it is accomplished via updates to the prediction algorithms and training, demonstrating that the core system's data (the facial video tensor) is rich enough to support multiple facets of health monitoring in a unified framework.

In contrast to prior rPPG pipelines that apply global color averaging and bandpass filtering, the disclosed hardware pipeline performs (i) ROI-mesh tracking with landmark-locked windows, (ii) OCT-style tensorization that treats time as a depth axis and fuses multi-channel physiological features {iPPG, iBCG, ∇xl, ∇yl, skin-mask}, and (iii) cross-modal phase-consistency constraints during training. This combination yields deterministic latency≤50 ms for 1.0 s input windows at ≥30 fps and sustains signal-quality indices≥0.90 during ±30° head rotation and 50-1000 lx illuminance change, representing a concrete improvement in the functioning of camera-based physiological sensing systems rather than an abstract data analysis.

An embodiment of the invention was evaluated in a prospective validation study comparing heart rate (HR), respiratory rate (RR), peripheral oxygen saturation (SpO₂), and blood pressure (BP) against clinically approved reference devices. The study reported: mean absolute difference for HR of 1.41 bpm (mean absolute percentage difference 1.69%); for RR, mean absolute difference 0.86 breaths/min (mean absolute percentage difference 4.72%); for SPO₂, mean absolute percentage difference 0.59%; and BP prediction accuracies of 94.81% (systolic) and 95.71% (diastolic) against the reference values. These results illustrate that the invention, operated without facial accessories and under normal illumination, met predefined clinical accuracy thresholds for all four vitals in that study. See: Dcruz J G, Yeh P. The Accuracy of Pulse Oxygen Saturation, Heart Rate, Blood Pressure, and Respiratory Rate Raised by a Contactless Telehealth Portal: Validation Study, JMIR Formative Research, 28 Jun. 2024; 8:e55361. doi:10.2196/55361.

### Exemplary Implementations and Embodiments

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases, it will be recognized that references to the “invention” will refer to subject matter recited in one or more, but not necessarily all, of the claims.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

Various embodiments are further described herein with reference to the accompanying figures. It should be noted that the description and figures relate to exemplary embodiments and should not be construed as a limitation to the subject matter of the present disclosure. It is also to be understood that various arrangements may be devised that, although not explicitly described or shown herein, embody the principles of the subject matter of the present disclosure. Moreover, all statements herein reciting principles, aspects, and embodiments of the subject matter of the present disclosure, as well as specific examples, are intended to encompass equivalents thereof. Yet further, for the sake of brevity, operation or working principles pertaining to the technical material that is known in the technical field of the present disclosure have not been described in detail so as not to unnecessarily obscure the present disclosure.

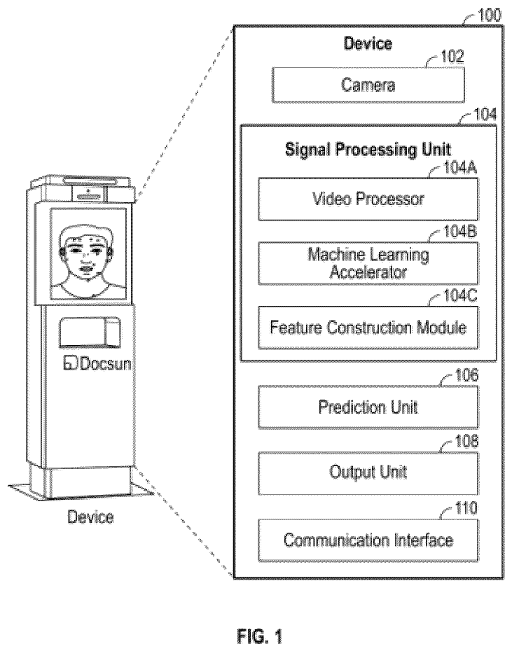

This detailed description provides an in-depth explanation of the device and method employed in the present invention for non-contact, non-invasive health monitoring. The device utilizes a high-resolution imaging unit to capture real-time video data of a user's face, processes this data to detect physiological signals, and employs advanced deep learning techniques to predict a wide range of vital signs and wellness parameters. Key components, their configurations, and interactions within the device will be described, referencing specific elements and functionality based on FIGS. 1 through 13.

As can be seen from FIG. 1, a health monitoring device (100) includes a camera (102), a signal processing unit (104), a prediction unit (106), an output unit (108), and a communication interface (110). Each of these modules interacts to perform the core functions of the invention, which range from image acquisition to real-time health assessment and alert generation.

The device (100) has an architecture that supports integration across various platforms, including mobile, automotive, fitness equipment, and telehealth. The device (100) operates in real time and utilizes federated learning to maintain user privacy while allowing for model updates. The camera (102) is configured to capture real-time image data of a user's face. This image data consists of a sequence of high-resolution frames recorded under controlled lighting conditions or dynamically adjusted exposure settings, ensuring consistent pixel intensity values across frames. These frames are then processed to extract temporal and spatial variations corresponding to physiological signals.

The signal processing unit (104) is operatively coupled to the camera to receive the sequence of frames. The signal processing unit incorporates a video processor (104A), implemented as dedicated hardware circuitry, which detects facial landmarks using predefined algorithms. These algorithms, stored in on-chip memory, may include Haar cascade classifiers or deep-learning-based facial landmark detection models, capable of identifying key reference points such as the corners of the eyes, edges of the nostrils, and corners of the mouth. Using these landmarks, the video processor isolates one or more regions of interest (ROIs) corresponding to specific anatomical regions such as the cheeks, forehead, and nose, which are known to exhibit minute pixel intensity variations associated with blood perfusion and micro-movements due to cardiovascular and respiratory activities.

The time-series data from the ROIs is forwarded to a machine learning accelerator (104B) implemented as a specialized integrated circuit. The machine learning accelerator (104B) is configured to apply a trained neural network model, custom-trained on annotated video datasets correlated with ground-truth physiological signals, such as electrocardiogram (ECG) and pulse oximetry data. This model extracts imaging photoplethysmography (iPPG) and imaging ballistocardiography (iBCG) signals from the temporal and spatial intensity variations within the ROI data, representing subtle changes in blood volume pulsations and facial micro-motions.

The extracted iPPG and iBCG signals are further processed in a feature construction module (104C) based on the principles of optical coherence tomography (OCT). This module (104C) combines the extracted physiological signals with facial image features derived from localized pixel intensity gradients within and around the ROI landmarks, capturing spatial patterns of intensity variations. The module (104C) constructs a high-dimensional feature representation in the form of a volumetric tensor. This tensor includes temporal progression across frames as one dimension, spatial coordinates of the ROI as two dimensions representing height and width, and additional feature channels representing iPPG, iBCG, and localized pixel intensity gradients. The feature construction module (104C) is implemented in hardware logic, designed to mitigate background noise and improve motion stability in dynamic environments.

The high-dimensional feature representation is transmitted to a prediction unit (106) comprising a hardware-based inference engine. The prediction unit (106) applies a second trained neural network model, designed using a combination of convolutional layers and Transformer layers with self-attention and positional encoding. This model specializes in analyzing the volumetric tensor by combining temporal, spatial, and physiological feature relationships to compute one or more physiological metrics, such as pulse rate, breathing rate, blood oxygen saturation (SpO2), blood pressure, and heart rate variability. The prediction model is trained to ensure an error margin within medically validated criteria, achieving an error percentage below five percent.

The results of the prediction are presented to the user through the output unit (108). The output unit (108) comprises a hardware-based display interface that provides real-time feedback on the computed physiological metrics, allowing the user immediate insight into their health status. Additionally, a probabilistic inference component in the output unit estimates an uncertainty metric for the prediction. This is achieved by applying a statistically grounded method, such as Bayesian inference using trained ensembles of models, providing a quantifiable confidence measure for the reliability of the prediction.

The device (100) also features the communication interface (110) implemented as a hardware module, enabling the transmission of the physiological metrics and associated uncertainty metrics to external devices or networked systems. The communication interface supports standardized protocols such as Wi-Fi, Bluetooth, or Ethernet, and formats the data to comply with healthcare data interchange standards like HL7 and FHIR protocols, ensuring seamless integration with electronic health record systems or cloud-based analytics platforms. This allows the device (100) to function as part of a broader health monitoring ecosystem, suitable for applications such as remote patient monitoring, vehicle-based health assessments, and real-time health analytics in clinical or home settings.

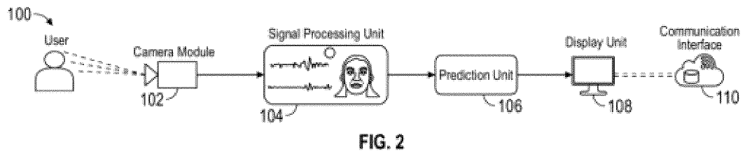

FIG. 2 represents the internal configuration and operation flow of the non-contact, non-invasive health monitoring device (100), detailing the interaction among its primary components. The camera (102) is positioned as the input unit, capturing real-time image data of the user's face. The camera ensures consistency in pixel intensity values by operating under controlled lighting conditions or employing dynamically adjusted exposure settings. The captured image data, comprising a sequence of high-resolution frames, is transmitted to the signal processing unit (104).

Within the signal processing unit, the video processor (104A) detects facial landmarks using predefined algorithms such as Haar cascade classifiers or deep learning models stored in on-chip memory. These landmarks serve as reference points for isolating regions of interest (ROIs) corresponding to anatomical areas like the cheeks, forehead, and nose. The isolated ROIs are analyzed to extract time-series data that captures physiological variations indicative of health metrics.

The time-series ROI data is forwarded to the machine learning accelerator (104B), which applies a trained neural network model. This model, designed specifically for extracting physiological signals, identifies imaging photoplethysmography (iPPG) and imaging ballistocardiography (iBCG) signals from the temporal and spatial intensity variations within the ROI data. These signals correspond to blood volume pulsations and micro-movements of facial tissues caused by cardiovascular and respiratory activity.

The feature construction module (104C) processes the extracted iPPG and iBCG signals along with localized pixel intensity gradients derived from the ROI landmarks. By combining these elements, the module creates a high-dimensional feature representation in the form of a volumetric tensor. This tensor encapsulates temporal progression, spatial coordinates, and feature channels representing physiological data, enabling robust analysis under various dynamic conditions.

The constructed tensor is then transmitted to the prediction unit (106), which contains a hardware-based inference engine. Using a trained neural network model composed of convolutional layers and Transformer architecture with self-attention, the prediction unit computes physiological metrics, including pulse rate, breathing rate, blood oxygen saturation (SpO2), and heart rate variability. These metrics are calculated with a medically validated accuracy, ensuring reliable health monitoring.

The output unit (108) displays the computed physiological metrics in real-time, offering immediate feedback to the user. Additionally, the probabilistic inference component estimates an uncertainty metric to provide a confidence measure for the predictions. This ensures that the user or medical practitioner is informed about the reliability of the data.

Finally, the communication interface (110) facilitates the transmission of the computed metrics and associated uncertainty data to external systems or devices. The interface supports standardized communication protocols such as Wi-Fi and Bluetooth and ensures data compatibility with electronic health record systems and cloud-based platforms through compliance with HL7 and FHIR standards. This integrated configuration enables seamless real-time health monitoring and data sharing for enhanced medical care and remote diagnostics.

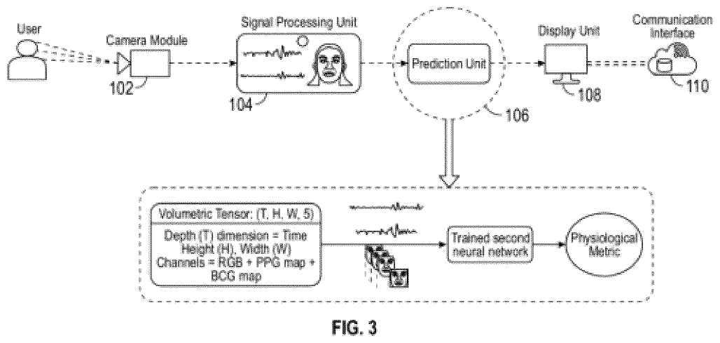

FIG. 3 illustrates the operational flow and interaction of the signal processing and analysis components within the non-contact, non-invasive health monitoring device (100). The diagram emphasizes the sequential processing of data captured by the camera (102) and its transformation into actionable physiological metrics.

The camera (102) serves as the initial data acquisition module, capturing real-time image data of the user's face under conditions that maintain consistent pixel intensity across frames. The captured image data is sent to the signal processing unit (104), where the video processor (104A) identifies facial landmarks using predefined algorithms. These landmarks delineate specific regions of interest (ROIs) corresponding to anatomical features such as the cheeks, forehead, and nose, which are known to exhibit physiological signals due to blood perfusion and micro-movements caused by cardiovascular and respiratory functions.

The video processor outputs time-series data from the ROIs, which is then processed by the machine learning accelerator (104B). The machine learning accelerator applies a neural network model trained on annotated datasets that correlate video data with ground-truth physiological signals like electrocardiograms (ECG) and pulse oximetry. This processing extracts imaging photoplethysmography (iPPG) and imaging ballistocardiography (iBCG) signals from temporal and spatial variations in the ROI data. The extracted signals reflect subtle physiological changes such as blood volume pulsations and micro-movements of the facial tissues.

The feature construction module (104C) receives the iPPG and iBCG signals and combines them with localized pixel intensity gradient features derived from the ROI landmarks. This integration forms a high-dimensional volumetric tensor that preserves temporal, spatial, and intensity-based physiological variations. The tensor's dimensions include temporal progression (time as a depth dimension), spatial coordinates representing the height and width of the ROI, and additional channels for iPPG, iBCG, and pixel intensity gradients.

The volumetric tensor is passed to the prediction unit (106), which performs advanced analysis using a second neural network model. This model, incorporating convolutional and Transformer-based layers with self-attention, analyzes the tensor to compute physiological metrics such as pulse rate, breathing rate, blood oxygen saturation (SpO2), and heart rate variability. The model ensures high accuracy and aligns with medically validated criteria.

The output unit (108) receives the computed physiological metrics and presents them to the user in real-time through a display interface. In parallel, a probabilistic inference component estimates uncertainty metrics for each prediction, providing a confidence measure for the reliability of the data. This enables informed decision-making by users or medical practitioners.

The communication interface (110) transmits the physiological metrics and their associated uncertainty data to external devices or network systems. This interface supports industry-standard communication protocols like Wi-Fi and Bluetooth and adheres to healthcare data interchange standards such as HL7 and FHIR. FIG. 3 thus showcases the streamlined and robust data flow from initial image capture to real-time physiological analysis and external data integration, highlighting the device's capability to deliver precise and reliable health monitoring.

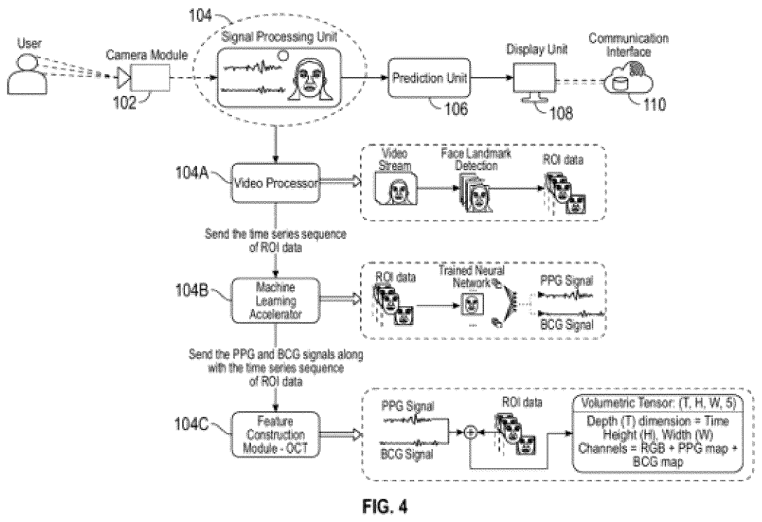

FIG. 4 illustrates the detailed processing workflow within the feature construction module (104C) and its interaction with other core components of the non-contact, non-invasive health monitoring device (100). This figure highlights how the module synthesizes physiological signals and facial image features into a unified, high-dimensional representation for further analysis.

The feature construction module (104C) receives input from the machine learning accelerator (104B) in the form of extracted imaging photoplethysmography (iPPG) and imaging ballistocardiography (iBCG) signals. These signals, derived from temporal and spatial intensity variations in the ROI data, represent physiological changes associated with blood flow and micro-movements in facial tissue. Simultaneously, the module incorporates localized pixel intensity gradient data computed from the ROI landmarks. These gradients capture spatial patterns of intensity variation around the landmarks, contributing critical information about the facial regions under analysis.

The module utilizes advanced principles of optical coherence tomography (OCT) to preprocess the incoming data, reducing background noise and motion artifacts. This ensures that the extracted signals and features remain stable and reliable, even under dynamic environmental conditions such as variable lighting or user motion. The preprocessed data is then combined to construct a volumetric tensor, which serves as a comprehensive representation of the physiological state captured over time.

The volumetric tensor is composed of multiple dimensions. The temporal progression of data frames is represented as the depth dimension, enabling the capture of time-dependent physiological changes. The spatial dimensions correspond to the height and width of the ROIs, preserving the anatomical structure and location-specific features. Additional feature channels within the tensor store the iPPG, iBCG, and pixel intensity gradient data, ensuring that both physiological and spatial information are fully integrated.

The constructed tensor is passed to the prediction unit (106), as indicated in FIG. 4, for further analysis. The seamless data flow from the feature construction module to the prediction unit ensures that the volumetric tensor retains its high-dimensional structure, enabling the neural network model in the prediction unit to accurately analyze complex temporal and spatial relationships. This modular workflow highlights the robust design of the device, where each processing stage contributes to the accuracy and reliability of the final physiological metrics.

FIG. 4 also underscores the scalability of the feature construction module, which is designed to adapt to various configurations and use cases. For instance, the module can handle data inputs from alternative imaging setups, such as dash cameras in vehicles or remote bedside monitoring devices, while maintaining consistent processing standards. This flexibility makes the module a critical component in extending the applicability of the device across diverse health monitoring environments.

Overall, FIG. 4 provides a comprehensive view of the feature construction module's operations and its central role in bridging raw data acquisition and advanced physiological analysis. By combining cutting-edge data preprocessing techniques with robust feature representation, the module ensures the device's ability to deliver precise and reliable health insights in real-time.

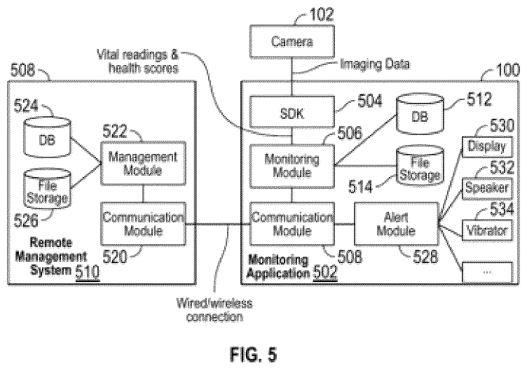

FIG. 5 illustrates an advanced non-contact, non-invasive health monitoring device (100) integrated with a monitoring application (502) and its associated components. At its core, the device (100) includes the camera (102) configured to capture real-time digital image data of a subject's face. This image data is processed through the signal processing unit (104), which houses the video processor (104A) for detecting facial landmarks and isolating regions of interest (ROIs) to generate a time-series sequence of data. The device (100) further includes the machine learning accelerator (104B) that applies a neural network model to extract imaging photoplethysmography (iPPG) and imaging ballistocardiography (iBCG) signals from the ROI data. These signals represent physiological characteristics such as blood volume pulsations and tissue micro-movements.

The feature construction module (104C) integrates the extracted iPPG and iBCG signals with localized pixel intensity gradients, constructing a high-dimensional volumetric tensor. This tensor combines temporal data, spatial ROI coordinates, and additional feature channels for advanced analysis. The tensor is passed to the prediction unit (106), which employs convolutional layers and Transformers to compute physiological metrics like pulse rate and oxygen saturation. Results are displayed in real-time via the output unit (108), which also incorporates a probabilistic inference component to estimate the reliability of predictions.

The monitoring application (502) complements this hardware device (100), integrating a Software Development Kit (SDK) (504) for customization, a monitoring module (506) for managing analytics, and a communication module (508) for data exchange with a remote management system (510).

The SDK (504) outputs the vital sign readings to the monitoring module (506). The monitoring module (506) can do active polling to acquire the readings and scores or passively receive the posted data of the readings and scores. The monitoring module (506) will save the readings and scores to a database (DB) (512). The DB (512) and file storage (514) are to keep the records and settings for the monitoring application (502).

The monitoring module (506) contains a logic to check if any reading or score is abnormal or meets the criteria to send an alert. The monitoring module (508) contains configurable alert criteria based on vital sign normal ranges. By default, an alert is triggered by an alert module (528) when any vital sign reading exceeds its normal physiological range. These ranges can be customized by users according to their specific monitoring needs. For example, the heart rate alert threshold could be adjusted for athletes who typically have lower resting heart rates compared to the general population. If sending an alert is required by the alert module (528), the monitoring module (506) will send a signal to the communication module (508) including the alert types to send an alert. The communication module (508) will communicate with the alert module (528) or the communication module (508) through a wired/wireless secure connection, such as HTTPS and/or WebSocket, with the remote management system (510) based on the alert types. The alert module (528) is in charge of sending local alerts such as showing an alert icon or text on the display module (530), playing a notification sound by a speaker (532), triggering a vibrator (534), showing a visual signal via a Light Emitting Diode (LED) light, and so on.

In an embodiment, the communication module (508) of the monitoring application (502) can communicate with a communication module (520) in the remote management system (510) and send over the information of the alert including the reading, score, and identification number of the device (100). In this embodiment, the management module (522) of the remote management system (510) can save the alert information to a database (DB) (524) and file storage (526), then show the alert on a user interface of the remote monitoring system (510). This allows for continuous remote supervision of the user's health, enabling timely intervention when required.

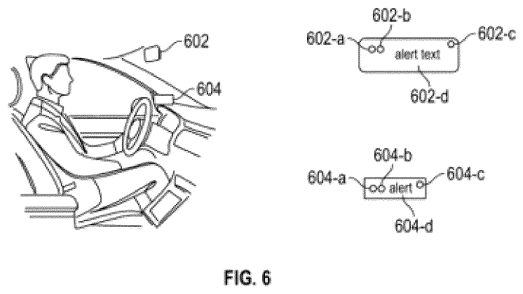

FIG. 6 shows the device (100) built in a car entertainment system, in accordance with an embodiment of the present disclosure. As can be seen from FIG. 6, the camera (102) can be installed at a back mirror (602), a device (604) on top of the dashboard, etc. In one embodiment, the back mirror (602) includes a camera (602-a), an infrared camera (602-b), an audio/light/vibrator alert component (602-c), and a visual text alert (602-d). In another embodiment, the device (604) on the top of the dashboard includes a camera (604-a), an infrared camera (604-b), an audio/light/vibrator alert component (604-c), and a visual text alert (604-d). In both embodiments, the cameras capture the driver's facial features and send them to the real-time SDK to process the signals. The real-time SDK responds to the driver's vital sign values and health scores for the device (100) to track the driver's body status. If the device (100) detects an abnormal situation such as uncomforted, stressed, or abnormal range of the vital signs, the device (100) can alert via visual/audio/vibration to the driver, or send a remote alert to the help center, or trigger the safety protection function of a car such as slowing down the speed, adjusting the temperature of an air conditioner, and so on to mitigate the risks or provide assistance. Alert criteria for driver monitoring can be customized by vehicle manufacturers or fleet operators to set specific thresholds for fatigue detection and stress levels based on their safety protocols.

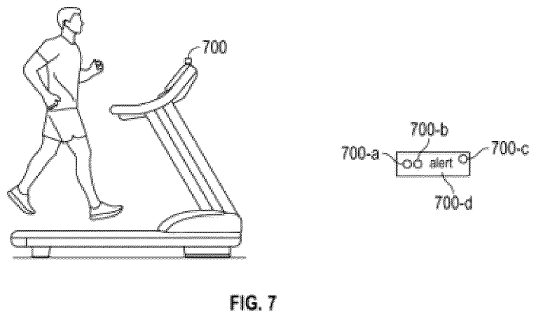

Referring to FIG. 7, the device (100) is integrated within an Advanced Driver Assistance System (ADAS). This setup leverages the real-time SDK and facial imaging data to monitor the physiological state of a driver, providing real-time feedback to enhance driving safety. By detecting signs of fatigue, stress, or other health abnormalities, the device (100) can intervene to mitigate risks associated with impaired driving, thereby reducing the likelihood of accidents caused by driver health issues. The device (100) operates through continuous data capture, analysis, and adaptive feedback mechanisms that interact with the vehicle's safety controls, offering a comprehensive solution for maintaining driver well-being.

FIG. 7 shows the device (100) is implemented as a device (700) built into gym equipment in accordance with an embodiment of the present disclosure. The device (700) is a device attached on top of, or embedded in the gym equipment. The device (700) includes a camera (700-a), an infrared camera (optional) (700-b), an audio/light/vibrator alert component (700-c), and a visual text alert (700-d). The cameras in the device (700) capture the facial features of the user and send them to the real-time SDK or API for analysis. The SDK will respond to vital sign values and health scores to the device (700) for tracking the user's workout and body status. The device (700) can alert via visual/audio/vibration or remote message to a gym help desk when a user is having abnormal issues to avoid overworking and causing injury. In an embodiment, gym equipment manufacturers can customize alert thresholds based on different workout intensity levels, user profiles, specific training programs, or user fitness levels.

Referring to FIG. 7, the device (700) is integrated within fitness monitoring equipment, such as a treadmill, to track the user's physiological metrics continuously during exercise routines. This setup enables the device (700) to provide real-time feedback on the user's physical status, alerting them to any significant changes in health metrics that may indicate overexertion, fatigue, or other health risks. By integrating directly into fitness equipment, the device (700) offers a seamless solution for exercise tracking, allowing users to optimize their workout intensity and ensure safe performance without the need for wearable devices. The device (700) operates by capturing facial imaging data, processing the data through the real-time SDK, and displaying results on the fitness equipment's interface, providing users with instant insights into their physiological state.

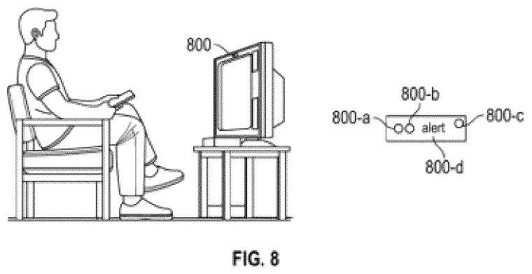

FIG. 8 shows the device (100) implemented as a device (800) built into a television set. The device (800) is attached on top of, or a component embedded in the television set. The device (800) includes a camera (800-a), an infrared camera (optional) (800-b), an audio/light/vibrator alert component (800-c), and a visual text alert (800-d). The cameras can be embedded in the television set or a set-top box and capture multiple users' facial features at the same time and send them to real-time SDK or API for analysis. The SDK or API will respond to the vital sign values and health scores of the device (800). The device (800) will keep track of the users' body status and send alerts to the users when there are any abnormal readings or scores to remind the user of further health checks based on the users' profiles. The device (800) can allow healthcare institutions to pre-set custom alert thresholds for different user groups segmented by the user's gender, age, and health conditions. While default alerts are triggered when readings fall outside normal physiological ranges, these thresholds can be adjusted based on individual user conditions and monitoring requirements.

Referring to FIG. 8, the device (800) is integrated into television sets, allowing it to perform non-invasive health monitoring in a home environment. This configuration transforms a common household device into a health and wellness station, enabling users to monitor key physiological metrics while they watch television or relax. The device (800) within the television provides a convenient and unobtrusive way for individuals and families to gain real-time health insights, enhancing awareness of overall wellness and enabling early detection of potential health issues. The television-integrated device (800) captures facial video data, processes it through the real-time SDK, and displays health metrics on-screen, providing users with seamless access to wellness information.

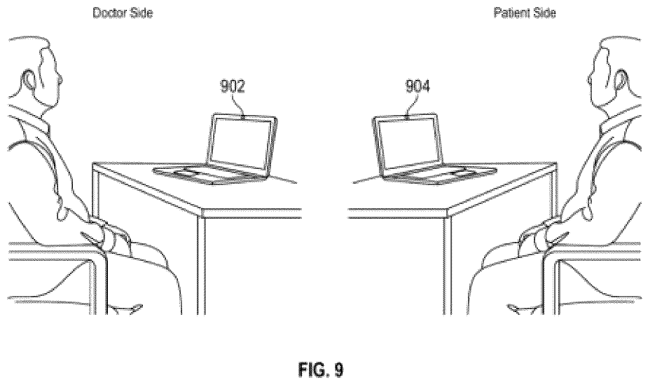

FIG. 9 shows a telehealth device (100) leveraging the computers on the doctor's and patient's sides to capture the patient's facial features in accordance with an embodiment of the present disclosure. In this embodiment, the cameras (902, 904) are implemented in a laptop or computer on the doctor's and patient's sides. The patient can perform a detection before entering a telehealth meeting with the doctor and the doctor will see the patient's vital sign readings and health scores. During the telehealth meeting, the doctor can issue another detection to trigger the detection on the patient's side to acquire the latest readings and scores. The device (100) will alert the doctor if the vital sign readings or health scores are beyond the thresholds set by the healthcare providers. The data will be stored in the telehealth device (100) for the doctor to track the patient's history. Healthcare providers can configure specific alert criteria for different patient conditions. The device (100) comes with default alert thresholds based on standard vital sign ranges, but these can be customized based on patient needs and medical protocols.

Referring to FIG. 9, the device (100) is integrated within telehealth devices to provide non-invasive remote health monitoring capabilities. This setup allows healthcare providers to assess a patient's vital signs and wellness metrics in real-time during virtual consultations. By capturing, processing, and analyzing facial video data, the device (100) offers healthcare professionals a continuous view of the patient's physiological state, enabling prompt and informed decision-making, especially for patients with chronic conditions or limited access to in-person healthcare. The device (100) supports a wide range of health metrics, including heart rate, respiratory rate, blood oxygen saturation (SpO2), and stress levels. By integrating telehealth applications, this setup enhances the quality of remote care and supports proactive health management.

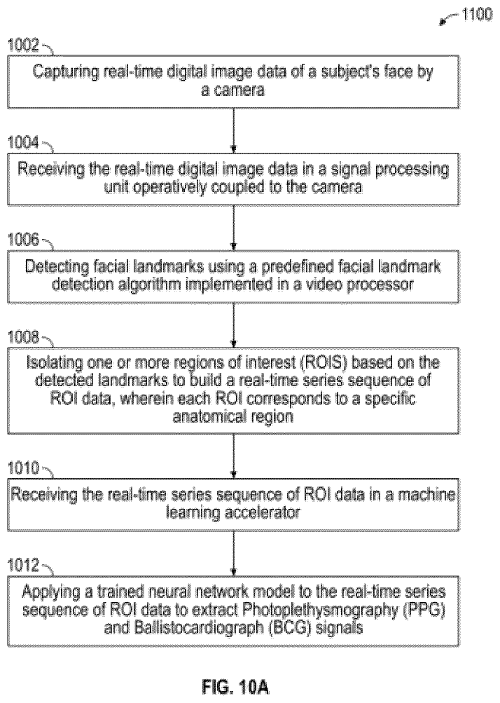

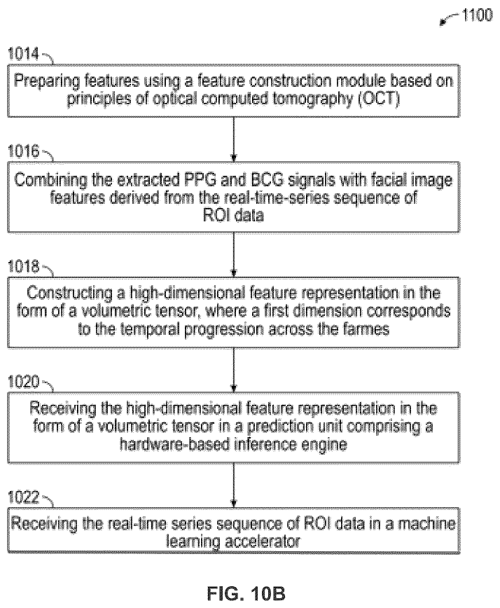

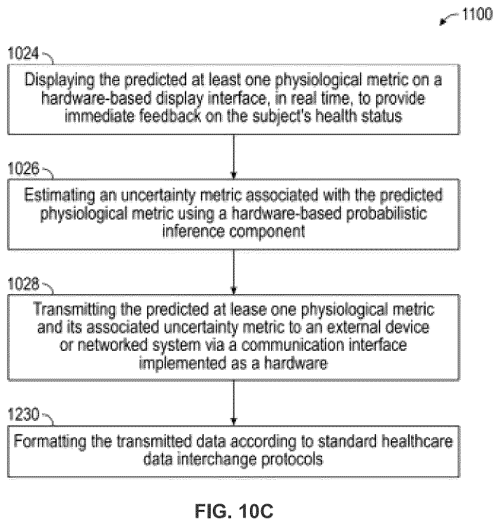

FIGS. 10A-10C illustrate an exemplary method (1000) for non-contact, non-invasive health monitoring, in accordance with an embodiment of the present disclosure. The order in which the method (1000) is described is not intended to be construed as a limitation, and any number of the described method blocks may be combined in any order to implement the methods, or an alternative method. Furthermore, method (1000) may be implemented by processing resource or computing device(s) through any suitable hardware, non-transitory machine-readable instructions, or a combination thereof.

It may also be understood that method (1000) may be performed by programmed computing devices (100) as depicted in FIGS. 1-9. Furthermore, the method (1000) may be executed based on instructions stored in a non-transitory computer-readable medium, as will be readily understood. The non-transitory computer-readable medium may include, for example, digital memories, magnetic storage media, such as one or more magnetic disks and magnetic tapes, hard drives, or optically readable digital data storage media. The method 1000 is described below with reference to computing device(s) as described above; other suitable devices for the execution of these methods may also be utilized. Additionally, the implementation of these methods is not limited to such examples.

At step 1002, the method (1000) begins with capturing real-time digital image data of a subject's face using a camera (102). The camera operates under controlled lighting conditions or dynamically adjusted exposure settings to ensure consistent pixel intensity values.

At step 1004, the real-time image data is received by a signal processing unit (104) that is operatively coupled to the camera (102).

At step 1006, with in the signal processing unit (104), a video processor (104A) implements a predefined facial landmark detection algorithm, identifying facial landmarks such as the corners of the eyes, edges of the nostrils, and corners of the mouth.

At step 1008, the facial landmarks are used to isolate one or more regions of interest (ROIs), corresponding to specific anatomical regions, such as the cheeks, forehead, and nose, which exhibit pixel intensity fluctuations related to physiological signals. This data is organized into a real-time series sequence of ROI data.

At step 1010, a machine learning accelerator (104B) processes this sequence of the ROI data.

At step 1012, the machine learning accelerator (104B) applies a trained neural network model to extract imaging photoplethysmography (iPPG) and imaging ballistocardiography (iBCG) signals from the ROI data.

At step 1014, a feature construction module (104C), based on principles of optical coherence tomography (OCT), prepares features.

At step 1016, the feature construction module (104C), based on principles of optical coherence tomography (OCT), prepares features by combining the extracted iPPG and iBCG signals with localized pixel intensity gradients derived from the ROI data. This integration captures spatial patterns of intensity variation (1114).

At step 1018, the prepared data is structured into a high-dimensional feature representation in the form of a volumetric tensor (1116). This tensor encompasses temporal progression across frames as a “depth” dimension, spatial coordinates representing ROI height and width, and additional feature channels for iPPG, iBCG, and localized pixel intensity gradients.

At step 1020, the high-dimensional feature representation is received in the form of a volumetric tensor in a prediction unit comprising a hardware-based inference engine.

At step 1022, the volumetric tensor is forwarded to a prediction unit (106), which employs a second neural network model comprising convolutional layers and Transformer architectures with self-attention mechanisms. This model analyzes the tensor to compute at least one physiological metric, such as pulse rate, blood oxygen saturation (SpO2), breathing rate, blood pressure, or heart rate variability (1120).

At step 1024, the prediction is displayed in real-time on a display module (116), providing immediate feedback on the subject's health status. The alert module (118) manages notifications, including visual alerts like icons or text, audible alerts via a speaker (816), tactile feedback using a vibrator (818), and visual signals through an LED light.

At step 1026, a probabilistic inference component estimates an uncertainty metric for each prediction, yielding a confidence measure based on variance estimates derived from a trained ensemble of models (1124).

At step 1028, finally, a communication interface (110) transmits the predicted physiological metrics and uncertainty metrics to an external system, such as a remote management system (510). The data is formatted in compliance with standard healthcare data interchange protocols, ensuring compatibility with electronic health record systems or cloud-based analytics platforms (1128). This method achieves a robust, efficient, and scalable solution for non-contact health monitoring, leveraging advanced hardware and software integration.

The method (1000) offers numerous advantages across clinical, fitness, and autonomous domains through its AI model insights, real-time processing capabilities, and privacy-preserving framework. These advantages ensure that the device proposed herein delivers both accurate health insights and seamless integration into various industries while safeguarding user data.

The AI model utilized in the invention significantly enhances health monitoring in clinical settings by offering a non-contact, non-invasive method to monitor critical vital signs such as include pulse rate, breathing rate, blood oxygen saturation (SpO2), blood glucose level, blood pressure, total cholesterol, heart rate variability, beta-ketones, and uric acid. By leveraging deep learning techniques and the combination of imaging photoplethysmography (iPPG) and imaging ballistocardiography (iBCG) signals, the device provides precise health metrics, reducing the margin of error typically associated with traditional contact-based monitoring devices.

The model's ability to emphasize BCG signals over iPPG signals ensures accurate predictions regardless of skin tone variations and motion artifacts, making it highly reliable for diverse patient populations. Additionally, the Bayesian Linear Layer offers the ability to predict not only vital sign values but also model uncertainty, providing healthcare professionals with confidence levels and reliability indicators for each prediction. This uncertainty estimation can guide clinical decisions, especially in scenarios where accurate real-time data is critical.

Furthermore, the AI model enables continuous monitoring, which is crucial for patients with chronic conditions such as cardiovascular diseases, diabetes, and respiratory disorders. The real-time nature of the model ensures that any sudden changes in a patient's health can be immediately detected, allowing for timely interventions and preventing adverse outcomes.

In the fitness domain, the AI model offers athletes and sports enthusiasts real-time insights into their physiological status, without the need for invasive wearables. The device's ability to predict vital metrics such as heart rate variability, metabolic state, and recovery times allows users to optimize their training routines and avoid overexertion.

Additionally, the AI model helps users track recovery by monitoring wellness parameters such as stress levels, immune health, and bone health. These wellness indicators enable athletes to tailor their fitness programs for maximum performance and recovery, reducing the risk of injury and ensuring balanced training. The continuous, non-invasive nature of the device provides users with uninterrupted feedback on their health, making it a valuable tool for maintaining peak physical performance.

The AI model integrates seamlessly with Advanced Driver Assistance Systems (ADAS) to ensure the safety and well-being of drivers. By analysing real-time video from driver-facing cameras, the device can predict signs of fatigue, stress, or illness that may impair driving ability. The ability to provide real-time feedback to the ADAS system allows for immediate intervention, such as issuing alerts or adjusting vehicle controls, thereby reducing the risk of accidents caused by driver health issues.

The model's fast processing speed and emphasis on iBCG signals ensure that accurate health insights are delivered even in dynamic environments like moving vehicles. This capability enhances the safety and performance of autonomous driving systems by continuously monitoring the driver's health and alerting them to any irregularities.

The Software Development Kit (SDK) and Application Programming Interface (API) are designed for real-time processing, offering developers an easy way to integrate the health monitoring device into various platforms, including mobile, automotive, fitness equipment, and telehealth applications. The SDK ensures that the real-time video data captured by the cameras is processed quickly and efficiently, delivering health insights within seconds. This enables immediate action based on the user's health condition, which is particularly useful in time-sensitive situations such as emergency healthcare or driver safety.

The SDK's real-time processing capability is crucial for applications requiring continuous monitoring. For instance, in fitness environments, the device can track a user's physiological responses throughout their workout session, providing instant feedback to help prevent overexertion. Similarly, in telehealth applications, real-time monitoring allows healthcare providers to assess a patient's condition during virtual consultations, ensuring up-to-date health information is available for diagnosis and treatment.

The SDK and API are built with privacy protection in mind. All data processing happens locally on the device, with the option to employ federated learning for AI model updates. This means that user data does not need to be transmitted to centralized servers for processing, significantly reducing the risk of data breaches and ensuring compliance with privacy regulations such as GDPR and HIPAA. By enabling local processing, the device ensures that sensitive health information remains on the user's device, offering users full control over their data.

Additionally, the federated learning approach allows the AI models to be updated over time without compromising user privacy. This ensures that the device continues to improve in accuracy and efficiency while keeping user data secure. The real-time SDK can automatically detect GPU availability or utilize WEBGL acceleration for enhanced processing power, ensuring that users experience fast, secure, and reliable health monitoring regardless of the platform they use.

By combining real-time processing with privacy-preserving techniques, the invention offers a highly secure health monitoring device that caters to various industries while maintaining the highest standards of data protection.

The above description does not provide specific details of the manufacture or design of the various components. Those of skill in the art are familiar with such details, and unless departures from those techniques are set out, techniques, known, related art or later developed designs and materials should be employed. Those in the art are capable of choosing suitable manufacturing and design details.

Note that throughout the disclosure, numerous references may be made regarding servers, services, engines, modules, interfaces, portals, platforms, or other devices formed from computing devices. It should be appreciated that the use of such terms is deemed to represent one or more computing devices having at least one processor configured to or programmed to execute software instructions stored on a computer-readable tangible, non-transitory medium also referred to as a processor-readable medium. For example, a server can include one or more computers operating as a web server, database server, or another type of computer server in a manner to fulfill described roles, responsibilities, or functions. Within the context of this document, the disclosed devices are also deemed to comprise computing devices having a processor and a non-transitory memory storing instructions executable by the processor that cause the device to control, manage, or otherwise manipulate the features of the devices.

It should be understood, however, that all of these and similar terms are to be associated with the appropriate physical quantities and are merely convenient labels applied to these quantities. Unless specifically stated otherwise, as apparent from the discussion herein, it is appreciated that throughout the description, discussions utilizing terms such as “capturing,” or “processing,” or “executing,” or “extracting,” “applying,” “generating,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The exemplary embodiment also relates to an apparatus for performing the operations discussed herein. This apparatus may be specially constructed for the required purposes, or it may comprise a general-purpose computer selectively activated or reconfigured by a computer program stored in the computer. Such a computer program may be stored in a computer-readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, or any type of media suitable for storing electronic instructions, and each coupled to a computer system bus.

Further, the terminology used herein is for the purpose of describing particular embodiments only and is not intended to be limiting to the disclosure. It would be appreciated if several of the above-disclosed and other features and functions, or alternatives thereof, could be combined into other devices or applications. Various presently unforeseen or unanticipated alternatives, modifications, variations, or improvements therein may subsequently be made by those skilled in the art without departing from the scope of the present disclosure as encompassed by the following claims.

The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

It will be appreciated that variants of the above-disclosed and other features and functions, or alternatives thereof, may be combined into many other different devices or applications. Various presently unforeseen or unanticipated alternatives, modifications, variations, or improvements therein may be subsequently made by those skilled in the art which are also intended to be encompassed by the following claims.

What is claimed is:

1. A non-contact, non-invasive physiological monitoring device, consisting of:
a camera configured to capture real-time facial image data of a subject as a sequence of frames under lighting conditions maintained at a fixed illuminance level within a range of 300 to 500 lux via artificial lighting;
a signal processing unit, operatively coupled to the camera, wherein said signal processing unit receives the sequence of frames from the camera, wherein the signal processing unit comprises:
a video processor implemented as a dedicated hardware circuitry, configured to:
detect facial landmarks using a predefined facial landmark detection algorithm; and
isolate one or more regions of interest (ROIs) based on the detected landmarks to build a time-series sequence of ROI data, wherein each ROI corresponds to a specific anatomical region;
a machine learning accelerator, implemented as a specialized integrated circuit, configured to:
receive the time-series sequence of ROI data from the video processor; and
apply a trained neural network model, trained using semi-supervised methods on annotated video datasets correlated with ground-truth physiological signals selected from the group consisting of, electrocardiogram (ECG), and pulse oximetry data, wherein the network architecture comprises transformer blocks with self-attention and positional-encoding mechanisms, enabling it to capture the spatio-temporal features of the ROI sequence and, in turn, detect subtle changes in pixel intensity linked to blood-volume pulsations and micro-motions of facial tissue that arise from temporal and spatial variations within the ROI time-series, wherein from these features, the model extracts imaging photoplethysmography (iPPG) and imaging ballistocardiography (iBCG) signals;
a feature construction module following the principles of optical coherence tomography (OCT) for feature preparation, providing improved background noise and motion reduction allowing better stability of feature extraction in dynamic environments of constant motion, wherein the feature construction module is implemented in hardware logic, configured to:
combine the extracted iPPG and iBCG signals from the machine learning accelerator, with facial image features derived from the time-series sequence of ROI data, wherein said facial image features comprise localized pixel intensity gradients calculated by comparing pixel values within and around the ROI landmarks, thus capturing spatial patterns of intensity variation; and
construct a high-dimensional feature representation in the form of a volumetric tensor, where:
one dimension corresponds to the temporal progression across the frames, effectively treating time as a “depth” dimension;
two dimensions represent the spatial coordinates of the ROI, capturing height and width; and
one or more additional feature channels represent iPPG, iBCG, alongside the localized pixel intensity gradients;
a prediction unit, comprising a hardware-based inference engine interfaced to the signal processing unit configured to:
receive the high-dimensional feature representation in the form of a volumetric tensor from the feature construction module; and
apply a second trained neural network model, the second trained neural network model is trained on facial video data and corresponding ground-truth physiological measurements, and is made up of a combination of Convolutional layers, and Transformer with self-attention with positional encoding, specialized in analysing the volumetric tensor, combining temporal, spatial, and physiological feature relationships of the iPPG, iBCG and ROI data of volumetric tensor, to compute at least one physiological metric, with significant improvement in performance with error percentage less than 5% that aligns with medically validated criteria;
an output unit comprising:
a hardware-based display interface configured to display the predicted at least one physiological metric from the prediction unit, in real-time to a user, providing immediate feedback on the subject's health status; and
a hardware-based probabilistic inference component configured to estimate an uncertainty metric associated with the predicted at least one physiological metric by applying a statistically grounded method, and inference using a trained ensemble of models that provides variance estimates, to yield a quantifiable confidence measure indicating the reliability of the prediction; and
a communication interface, implemented as a hardware module, configured to:
transmit the predicted at least one physiological metric and its associated uncertainty metric to an external device or networked system via a standardized communication protocol; and
format the transmitted data following standard healthcare data interchange protocols for ensuring compatibility with electronic health record systems or cloud-based analytics platforms, wherein said sequence of frames is recorded under controller lighting conditions maintained at a fixed illuminance level within a range of 300 to 500 lux to ensure consistent pixel intensity values by using artificial lighting, wherein said predefined facial landmark detection algorithm is selected from a group consisting of, a Haar cascade classifier, and a deep learning based facial landmark model, stored in on-chip memory, the algorithm identifying reference points such as corners of the eyes, edges of the nostrils, and corners of the mouth which is used to select ROIs from regions including the forehead, cheeks, and nose, which are known to exhibit minute pixel intensity fluctuations correlated to blood perfusion and micro-movements induced by the cardiovascular and respiratory activity, to optimize detection of hemodynamic variation, wherein said standardized communication protocol is selected from a group consisting of, Wi-Fi, Bluetooth, or Ethernet, wherein said standard healthcare data interchange protocols are selected from a group consisting of, HL7, FHIR protocols, ensuring compatibility with electronic health record systems or cloud-based analytics platforms.

2. A non-contact, non-invasive physiological monitoring device, comprising:
a camera configured to capture real-time facial image data of a subject as a sequence of frames under lighting conditions maintained at a fixed illuminance level within a range of 300 to 500 lux via artificial lighting;
a signal processing unit, operatively coupled to the camera, wherein said signal processing unit receives the sequence of frames from the camera, wherein the signal processing unit comprises:
a video processor implemented as a dedicated hardware circuitry, configured to:
detect facial landmarks using a predefined facial landmark detection algorithm; and
isolate one or more regions of interest (ROIs) based on the detected landmarks to build a time-series sequence of ROI data, wherein each ROI corresponds to a specific anatomical region;
a machine learning accelerator, implemented as a specialized integrated circuit, configured to:
receive the time-series sequence of ROI data from the video processor; and
apply a trained neural network model, trained using semi-supervised methods on annotated video datasets correlated with ground-truth physiological signals selected from the group consisting of, electrocardiogram (ECG), and pulse oximetry data, wherein the network architecture comprises transformer blocks with self-attention and positional-encoding mechanisms, enabling it to capture the spatio-temporal features of the ROI sequence and, in turn, detect subtle changes in pixel intensity linked to blood-volume pulsations and micro-motions of facial tissue that arise from temporal and spatial variations within the ROI time-series, wherein from these features, the model extracts imaging photoplethysmography (iPPG) and imaging ballistocardiography (iBCG) signals;
a feature construction module following the principles of optical coherence tomography (OCT) for feature preparation, providing improved background noise and motion reduction allowing better stability of feature extraction in dynamic environments of constant motion, wherein the feature construction module is implemented in hardware logic, configured to:
combine the extracted iPPG and iBCG signals from the machine learning accelerator, with facial image features derived from the time-series sequence of ROI data, wherein said facial image features comprise localized pixel intensity gradients calculated by comparing pixel values within and around the ROI landmarks, thus capturing spatial patterns of intensity variation; and
construct a high-dimensional feature representation in the form of a volumetric tensor, where:
one dimension corresponds to the temporal progression across the frames, effectively treating time as a “depth” dimension;
two dimensions represent the spatial coordinates of the ROI, capturing height and width; and
one or more additional feature channels represent iPPG, iBCG, alongside the localized pixel intensity gradients;
a prediction unit, comprising a hardware-based inference engine interfaced to the signal processing unit configured to:
receive the high-dimensional feature representation in the form of a volumetric tensor from the feature construction module; and
apply a second trained neural network model, the second trained neural network model is trained on facial video data and corresponding ground-truth physiological measurements, and is made up of a combination of Convolutional layers, and Transformer with self-attention with positional encoding, specialized in analysing the volumetric tensor, combining temporal, spatial, and physiological feature relationships of the iPPG, iBCG and ROI data of volumetric tensor, to compute at least one physiological metric, with significant improvement in performance with error percentage less than 5% that aligns with medically validated criteria;
an output unit comprising:
a hardware-based display interface configured to display the predicted at least one physiological metric from the prediction unit, in real-time to a user, providing immediate feedback on the subject's health status; and
a hardware-based probabilistic inference component configured to estimate an uncertainty metric associated with the predicted at least one physiological metric by applying a statistically grounded method, and inference using a trained ensemble of models that provides variance estimates, to yield a quantifiable confidence measure indicating the reliability of the prediction; and
a communication interface, implemented as a hardware module, configured to:
transmit the predicted at least one physiological metric and its associated uncertainty metric to an external device or networked system via a standardized communication protocol; and
format the transmitted data following standard healthcare data interchange protocols for ensuring compatibility with electronic health record systems or cloud-based analytics platforms.

3. The device of claim 2, wherein said sequence of frames is recorded under controller lighting conditions maintained at a fixed illuminance level within a range of 300 to 500 lux to ensure consistent pixel intensity values by using artificial lighting.

4. The device of claim 3, wherein said predefined facial landmark detection algorithm is selected from a group consisting of, a Haar cascade classifier, and a deep learning based facial landmark model, stored in on-chip memory, the predefined facial landmark detection algorithm identifying reference points such as corners of the eyes, edges of the nostrils, and corners of the mouth which is used to select ROIs from regions including the forehead, cheeks, and nose, which are known to exhibit minute pixel intensity fluctuations correlated to blood perfusion and micro-movements induced by the cardiovascular and respiratory activity, to optimize detection of hemodynamic variation.

5. The device of claim 2, wherein said predefined facial landmark detection algorithm is selected from a group consisting of, a Haar cascade classifier, and a deep learning based facial landmark model, stored in on-chip memory, the predefined facial landmark detection algorithm identifying reference points such as corners of the eyes, edges of the nostrils, and corners of the mouth which is used to select ROIs from regions including the forehead, cheeks, and nose, which are known to exhibit minute pixel intensity fluctuations correlated to blood perfusion and micro-movements induced by the cardiovascular and respiratory activity, to optimize detection of hemodynamic variation.

6. The device of claim 5, wherein said standardized communication protocol is selected from a group consisting of, Wi-Fi, Bluetooth, or Ethernet.

7. The device of claim 2, wherein said standardized communication protocol is selected from a group consisting of, Wi-Fi, Bluetooth, or Ethernet.

8. The device of claim 7, wherein said standard healthcare data interchange protocols are selected from a group consisting of, HL7, FHIR protocols, ensuring compatibility with electronic health record systems or cloud-based analytics platforms.

9. The device of claim 2, wherein said standard healthcare data interchange protocols are selected from a group consisting of, HL7, FHIR protocols, ensuring compatibility with electronic health record systems or cloud-based analytics platforms.

10. A method for non-contact, non-invasive monitoring of physiological well-being, comprising:
capturing a real-time video stream of a subject's face by a camera;
detecting facial landmarks from the video stream by a hardware-based video processor;
extracting one or more regions of interest (ROIs) corresponding to physiological zones from the detected landmarks to create a time-series ROI dataset;
processing the ROI dataset with a first neural network model applied by a machine learning accelerator to extract photoplethysmography (iPPG) and ballistocardiography (iBCG) signals;
generating a volumetric tensor by a feature construction module that combines iPPG, iBCG, and pixel intensity gradients across spatial and temporal dimensions;
analyzing the volumetric tensor by a second neural network comprising convolutional and transformer layers to compute at least one physiological metric;
estimating a confidence score corresponding to the predicted metric by a probabilistic inference engine; and
displaying the physiological metric in real-time and transmitting it to an external platform via healthcare-compliant data protocols.

11. The method of claim 10, wherein the video stream is acquired under dynamically controlled illumination to ensure uniform intensity distribution across facial pixels.

12. The method of claim 11, wherein the first neural network is trained with ground-truth data including electrocardiogram (ECG) and pulse oximetry references to enhance signal extraction accuracy.

13. The method of claim 10, wherein the first neural network is trained with ground-truth data including electrocardiogram (ECG) and pulse oximetry references to enhance signal extraction accuracy.

14. The method of claim 13, wherein the feature construction module applies optical coherence tomography (OCT)-inspired spatial stacking to improve background noise suppression and motion robustness.

15. The method of claim 14, wherein the feature construction module applies optical coherence tomography (OCT)-inspired spatial stacking to improve background noise suppression and motion robustness.

16. The method of claim 15, wherein the second neural network applied in analysis includes temporal self-attention mechanisms to capture long-range signal dependencies across frames.

17. The method of claim 16, wherein the predicted physiological metric is selected from the group consisting of: blood pressure, stress index, immune readiness, metabolic health score, or oxygen saturation.

18. The method of claim 10, wherein the predicted physiological metric is selected from the group consisting of: blood pressure, stress index, immune readiness, metabolic health score, or oxygen saturation.

19. The method of claim 18, further comprising the step of triggering a remote alert if the physiological metric exceeds a predefined threshold indicating an emergency condition.

20. The method of claim 10, further comprising the step of triggering a remote alert if the physiological metric exceeds a predefined threshold indicating an emergency condition.
